(19) United States
(12) Patent Application Publication
(10) Pub. No.: US 2023/0340546 A1
(43) Pub. Date: Oct. 26, 2023

(54) USE OF UV-ACTIVATED ENZYMES TO IMPLEMENT OXIDATION REACTIONS AND THE CORRESPONDING PROCESSES

(71) Applicants: INSTITUT NATIONAL DE LA RECHERCHE POUR L'AGRICULTURE, L'ALIMENTATION ET L'ENVIRONNEMENT, Paris (FR); CENTRE NATIONAL DE LA RECHERCHE SCIENTIFIQUE, Paris (FR); UNIVERSITE D'AIX MARSEILLE, MARSEILLE CEDEX 07 (FR); ECOLE CENTRALE DE MARSEILLE, Marseille (FR)

(72) Inventors: Bastien BISSARO, Marseille (FR); Jean-Guy BERRIN, Roquevaire (FR); Michael LAFOND, Le Pradet (FR)

(73) Assignees: INSTITUT NATIONAL DE LA RECHERCHE POUR L'AGRICULTURE, L'ALIMENTATION ET L'ENVIRONNEMENT, Paris (FR); CENTRE NATIONAL DE LA RECHERCHE SCIENTIFIQUE, Paris (FR); UNIVERSITE D'AIX MARSEILLE, MARSEILLE CEDEX 07 (FR); ECOLE CENTRALE DE MARSEILLE, Marseille (FR)

(21) Appl. No.: 18/005,079

(22) PCT Filed: Jul. 21, 2021
(86) PCT No.: PCT/EP2021/070365

§ 371 Date: Jan. 11, 2023

(30) Foreign Application Priority Data
Jul. 21, 2020 (EP) 20186971.6

(51) Int. Cl. C12P 7/22; C12N 9/04

(52) U.S. Cl. CPC C12P 7/22; C12N 9/0006; C12Y 101/03013; C12Y 101/03009; C12Y 101/03007

(57) ABSTRACT

The use of UV-activated Copper Radical Oxidase (CRO) enzymes in the implementation of oxidation reactions. Also, a process for oxidizing organic compounds using enzymes which are activated by UV light. The process also leads to concomitant formation of hydrogen peroxide, that can optionally be used in hydrogen peroxide mediated processes. Further, the process relates to the oxidation of alcohols in aldehydes.

The invention relates to the use of UV-activated enzymes to implement oxidation reactions of organic compounds. The present invention also relates to a process for oxidizing organic compounds using enzymes which are activated by UV light.

The development of environmentally friendly processes in the chemical industry is of ongoing concern. In this respect, the use of enzymes in the production of chemicals has been explored in the past. One such class of enzymes are Copper Radical oxidases (CROs), that can catalyze a wide variety of oxidation reactions. These enzymes comprise a Copper ion in their active site, hence their name Most CRO-enzymes belong to the CAZy family AA5 (Auxiliary Activity n°5 family), which encompasses CAZy subfamily 1 (AA5_1) and CAZy subfamily 2 (AA5_2).

These CRO enzymes however need to be activated prior, or during the intended use in catalysis. Such activation is typically achieved by adding a redox-activator such as Horseradish peroxidase (HRP) to the reaction mixture comprising the enzyme. Horseradish peroxidase is an expensive and relatively unstable material, that, in addition, must be removed from the reaction mixture during the purification of the reaction product.

Hence the need for activating these enzymes using different methods, in particular methods that do not imply adding an exogeneous redox-activator.

Thus, one aim of the present invention is the use of UV-activated enzymes in the oxidation of an organic compound.

Another aim of the present invention is the activation of specific enzymes using UV light.

Yet another aim of the present invention is to provide a process for the oxidation of chemical compounds, which can be controlled, without the use of exogeneous redox-activators.

Yet another aim of the present invention is to provide control over said process.

Yet another aim of the present invention is the use of UV-activated enzymes in the production of hydrogen peroxide.

Yet another aim of the present invention is the use of the hydrogen peroxide thus produced.

Thus, a first object of the present invention concerns the use of UV-activated Copper Radical Oxidase (CRO) enzymes for the implementation of a chemical oxidation process of an organic compound.

The inventors have surprisingly found that Copper Radical Oxidase enzymes can be activated by UV light. This feature has not been described in the literature to date and allows for the use of these enzymes in oxidation reaction, without the need to add external redox activators.

Without being bound to theory, the mechanism of the UV-activation of CRO enzymes implies the formation of a radical on a Tyrosine residue that is located close to the copper that is present in the active site of the enzyme.

This was corroborated by the inventors in an EPR (Electron Paramagnetic Resonance) experiment, the results of which are shown in FIG. 1.

Irradiation of the enzyme with UV light shows the appearance of 2 signals (FIG. 1B), as compared to the non-irradiated enzyme (FIG. 1A). These appearing signals are characteristic of radicals located in the proximity of the copper ion.

CRO-enzymes can exist in a “mature” form, and in a “non-mature” form. Non-mature enzymes are inactive towards the catalytic oxidation reactions according to the present invention.

For the UV-activation to happen, the enzyme should first be “matured”, implying the copper mediated formation of a thioether bond between a cysteine residue and a Tyrosine residue in the enzymes active site (Ito et al., Novel Thioether Bond Revealed by a 1.7 A Crystal Structure of Galactose Oxidase, Nature 1991 Mar. 7; 350(6313):87-90, and Firbank et al., Crystal structure of the precursor of galactose oxidase: An unusual self-processing enzyme, Proc Natl Acad Sci USA. 2001 Nov. 6; 98(23): 12932-12937).

In most cases, this maturation step occurs naturally during the production of the enzyme, and the isolated CRO enzyme is already mature. However, in rare cases, it is possible to achieve maturation in vitro. For instance, when enzyme production is carried out under metal-free conditions. The result is a precursor of the CRO-enzyme that does not have copper in the active site. This isolated precursor enzyme can subsequently be treated with a copper source, allowing for in vitro maturation (Roger et al., J. Am. Chem. Soc. 2000, 122, 5, 990-991).

Scheme 1 shows the postulated catalytic cycle of the oxidation of a compound using a CRO enzyme.

A CRO enzyme in inactive form (semi-reduced), is exposed to UV light, thereby forming a CRO enzyme in active form (fully oxidized). This active species is able to oxidize a compound into an oxidized product, resulting in the concomitant formation of a fully reduced CRO enzyme. Said fully reduced enzyme subsequently reduces oxygen into hydrogen peroxide, thus regenerating the fully oxidized active form that can be involved in another reaction cycle. Turnover numbers of more than 10,000 can thus be achieved.

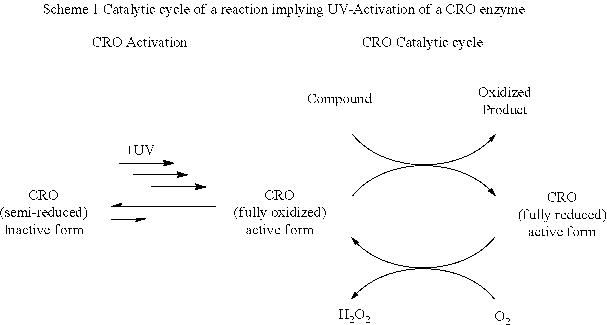

Thus, the expression “UV-activated” refers to control of the catalytic activity of an CRO enzyme by application of UV light as described herein. The enzyme is “activated” when UV light applied to the enzyme causes structural changes in the enzyme, in particular the formation of radical species, such as the formation of a radical on a Tyrosine residue that is located close to the copper that is present in the active site of the enzyme.

In other words, the CRO enzymes are capable of being controlled by UV light to be active, or more active, with respect to their catalytic activity towards oxidation reactions as disclosed herein.

The UV activated state of the enzyme can be shown by performing a use-test. Thus, for example, improved kinetics of the oxidation of benzyl alcohol into benzaldehyde is observed when the oxidation is catalyzed by a UV activated CRO enzyme, as compared to a non-UV activated CRO enzyme.

The UV activated state of the enzyme can also be determined by analysis of the presence of the Tyrosine radical, for instance by EPR ((Electron Paramagnetic Resonance).

According to another embodiment, the present invention concerns the use of UV-activated Copper Radical Oxidase (CRO) enzymes as described above, wherein the chemical oxidation of the organic compound is followed by the formation of hydrogen peroxide.

Thus, according to this embodiment, UV-activated Copper Radical Oxidase (CRO) enzymes are used for the implementation of a chemical oxidation process of an organic compound, and are used for the implementation of a reduction process of oxygen to hydrogen peroxide.

In a preferred embodiment of the present invention; the formation of hydrogen peroxide is concomitant.

The present invention also concerns the use of UV-activated Copper Radical Oxidase (CRO) enzymes for the implementation of a chemical oxidation process of an organic compound, in particular, wherein the chemical oxidation of the organic compound is followed by the formation of hydrogen peroxide.

A second object of the present invention concerns a process for the chemical oxidation of an organic compound,
said process comprising the step of contacting an organic compound bearing an oxidizable function with at least one Copper-Radical Oxidase (CRO) enzyme in the presence of molecular oxygen,
wherein said at least one CRO enzyme is activated by a step of exposing said at least one CRO enzyme to UV light, to obtain a UV-activated CRO enzyme,
whereby the organic compound is oxidized into an oxidized organic product, and whereby hydrogen peroxide is generated.

According to the present invention, the expression “oxidizable function” relates to a functional group able to be oxidized, in particular a carbon-heteroatom single bond, that is oxidized into a carbon heteroatom double bond, wherein the heteroatom preferably chosen from O, N, or S, the oxidizable function is in particular a —C—OH group that is oxidized into a —C═O group. The oxidizable organic compounds according to the present invention comprise at least one oxidizable function as defined above.

According to the present invention, a step of contacting an organic compound with at least one CRO-enzyme, refers to bringing together said organic compound with said at least one CRO-enzyme. The organic compound thus is “in contact” with the at least one CRO-enzyme, and thus becomes available to the said at least one CRO-enzyme, allowing them to interact.

The organic compound and the at least one CRO-enzyme are typically brought in solution together, in the same reaction flask.

According to the present Invention, the expression “at least one CRO-enzyme” refers to one or more than one CRO-enzymes of different sequence, in particular from 1 to 3 CRO enzyme(s), more in particular 1, 2 or 3 CRO-enzyme(s).

For example, in a step of contacting an organic compound with 2 CRO-enzymes, a mixture of galactose oxidase and an alcohol oxidase, or a mixture of 2 galactose oxidases of different sequence, is used.

According to the present invention, “UV light” is a form of electromagnetic radiation having a wavelength ranging from 200 to 400 nm.

With “200 to 400 nm” is also meant the following ranges: 200 to 350 nm, 200 to 300 nm, 200 to 250 nm, 200 to 225 nm, 240 to 400 nm, 300 to 400 nm, 350 to 400 nm, 225 to 375 nm, 240 to 350 nm, 240 to 320 nm, and 270 to 290 nm.

Below 200 nm harmful species can be generated, resulting in side-reactions.

According to the present invention, the expression “exposing said at least one CRO enzyme to UV light” indicates that the CRO-enzyme is irradiated with UV light, whereby the UV light reaches the enzyme. In this respect, an experimental setup can be a UV-lamp that is immersed into the solution comprising the enzyme. Alternatively, the UV-source can be placed outside the reactor that contains the solution comprising the enzyme, said reactor being made of a material that does not block the UV light, such as Quartz. The UV-source can be artificial, such as a lamp, or natural, such as sun-light.

The exposure of the at least one CRO enzyme to UV light, during the step of exposing said at least on CRO enzyme with UV light, can occur at different stages of the process.

Thus, according to a preferred embodiment, the present invention relates to a process for chemical oxidation as previously described, wherein the step of exposing said at least one CRO enzyme to UV light is carried out during the step of contacting.

Thus, according to another preferred embodiment, the present invention concerns a process for chemical oxidation as previously described, wherein the step of exposing said at least one CRO enzyme to UV light is carried out before the step of contacting.

In this embodiment, the at least one CRO enzyme is exposed to UV light before the step of contacting with the organic compound to be oxidized. In other word, the enzyme is pre-exposed leading to a pre-activated enzyme.

Said pre-exposing the enzyme to UV light is preferably carried out by solubilizing the enzyme in a reaction solvent and providing UV light. Once activation is achieved, which can be analyzed using an EPR analysis, the enzyme is contacted with the organic compound.

Thus, according to another preferred embodiment, the present invention concerns a process for chemical oxidation as previously described, wherein the step of exposing said at least one CRO enzyme to UV light is carried out both before and during the step of contacting.

In this embodiment, the enzyme is pre-exposed to UV light, and is also exposed to UV light during the contacting step.

According to another preferred embodiment, the present invention concerns a process for chemical oxidation as previously described, wherein the step of exposing said at least one CRO enzyme to UV light is carried out
during the step of contacting,
before the step of contacting, or
both before and during the step of contacting,
   in particular wherein said step of exposing to UV light before the step of contacting is maintained for 1 second to about 10 minutes, followed by contacting said UV-activated enzyme with said organic compound.

According to another preferred embodiment, the present invention concerns a process for chemical oxidation as described above,
wherein during the contact of said organic compound with said enzyme, the exposure to UV light is continuous or intermittent, preferably intermittent.

In some embodiments, the exposure to UV light is continuous, meaning uninterrupted exposure to UV light, whereby the exposure is maintained during the entire course of the reaction.

In other embodiments, the exposure to UV light is intermittent, whereby said exposure is interrupted by one or more periods of non-exposure. For example, by switching the light source on and off.

As shown in the postulated reaction mechanism of Scheme 1, the fully oxidized active form can be converted into a semi-reduced form, leading to an inactive enzyme that should be re-exposed to UV light in order to be activated.

Thus, by continuously exposing the enzyme to UV light during the oxidation reaction, the enzyme is continuously regenerated in situ.

On the contrary, by interrupting the exposure to UV light, the enzyme can become semi-reduced and inactive, and a decrease in catalytic activity is observed.

Subsequent renewed UV light exposure leads to reactivation of the enzyme.

This feature of the invention allows for control over the reaction kinetics, in a sense by switching the enzyme activity on and off. This feature also allows, if desired, to perform reactions in a sequential manner.

According to another preferred embodiment, the present invention concerns a process for chemical oxidation as described above,
wherein during the contact of said organic compound with said enzyme, the exposure to UV light is intermittent,
and preferably is maintained for one or more periods of 1 second to 1 hour, in particular 1 second to 10 minutes, followed by one or more non-exposure periods of 1 second to 1 hour.

The exposure periods may be of equal length, or may be of different length than the non-exposure periods.

In case of more than one 1 exposure period, the subsequent exposure periods may be of equal length or may be of different length.

In case of more than one 1 non-exposure period, the subsequent non-exposure periods may be of equal length or may be of different length.

By “one or more periods” should also be understood, 1 to 1000 periods, 1 to 500 periods; 1 to 250 periods, 1 to 100 periods, 1 to 10 periods, 10 to 1000 periods, 100 to 1000 periods, 250 to 1000 periods, 500 to 1000 periods, 10 to 500 periods, or 100 to 250 periods. These ranges include all individual integer values, so that for example “1 to 10 periods” has the meaning of 1, 2, 3, 4, 5, 6, 7, 8, 9, or 10 periods.

By “1 second to 1 hour” should be understood all individual time-values comprised within this range, so that for example “1 second to 10 seconds” includes 1, 2, 3, 4, 5, 6, 7, 8, 9, and 10 seconds.

By “1 second to 1 hour” should in particular be understood 1 second to 45 minutes, 1 second to 30 minutes, 1 second to 15 minutes, 1 second to 10 minutes, 1 second to 5 minutes, 1 second to 2 minutes, 1 to 60 seconds, 1 to 50 seconds, 1 to 40 seconds, 1 to 30 seconds, 1 to 20 seconds, 1 to 10 seconds, 1 to 5 seconds, 1 minute to 1 hour, 10 minutes to 1 hour, 30 minutes to 1 hour, 1 minute to 30 minutes, or 5 minutes.

According to another preferred embodiment, the present invention concerns a process for chemical oxidation as described above,
wherein during the contact of said organic compound with said enzyme, the exposure to UV light is continuous or intermittent,
in particular, wherein during the contact of said organic compound with said enzyme, the exposure to UV light is intermittent, and preferably is maintained for one or more periods of 1 second to 1 hour, followed by one or more non-exposure periods of 1 second to 1 hour.

According to another preferred embodiment, the present invention concerns a process for chemical oxidation as described above,
wherein the UV light has a wavelength comprised from 240 to 320 nm, preferably from 270 to 290 nm, more preferably has a wavelength of about 280 nm.

According to another preferred embodiment, the present invention concerns a process for chemical oxidation as described above,
wherein, during the step of exposing said at least one CRO enzyme to UV light, the enzyme is exposed to UV light having a light intensity comprised from 0.01 to 1000 mW/cm², in particular from 1 to 100 mW/cm².

According to the present invention, it should be understood that the light intensity values correspond to the light intensity of the UV light to which the enzyme is exposed, before activation of said enzyme in other words, of the UV light that reaches the reaction medium. Said light intensity can be different from the light intensity of the UV light emitted from the UV-source.

According to another preferred embodiment, the present invention concerns a process for chemical oxidation as described above,
wherein, during the step of exposing said at least one CRO enzyme to UV light, the enzyme is exposed to of from 1 to 100 μmol photon·s⁻¹·m⁻²; in particular of from 10 to 30 μmol photon·s⁻¹·m².

With “1 to 100 μmol photon·s⁻¹·m⁻²” is also meant the following ranges: 1 to 75 μmol photon·s⁻¹·m², 1 to 50 μmol photon·s⁻¹·m⁻², 1 to 25 μmol photon·s⁻¹·m⁻², 1 to 15 μmol photon·s⁻¹·m⁻², 10 to 100 μmol photon·s⁻¹·m⁻², 25 to 100 μmol photon·s⁻¹·m⁻², 50 to 100 μmol photon·s⁻¹·m⁻², 75 to 100 μmol photon·s⁻¹·m⁻², 5 to 50 μmol photon·s⁻¹·m⁻², 10 to 50 μmol photon·s⁻¹·m⁻², in particular 10 to 30 μmol photon·s⁻¹·m⁻², and more in particular 14 to 15 μmol photon·s⁻¹·m⁻², or about 14.6 μmol photon·s⁻¹·m⁻².

According to another preferred embodiment, the present invention concerns a process for chemical oxidation as described above,
wherein oxygen is constantly or discontinuously provided during the contact of said organic compound with said enzyme.

When oxygen is constantly provided, the provision of oxygen is uninterrupted, whereby oxygen provision is maintained during the entire course of the reaction.

Discontinuous provision of oxygen means that periods of oxygen provision are altered with periods of non-provision of oxygen. The oxygen provision is thus interrupted by one or more periods of non-provision. For example, by stopping the bubbling of oxygen into the reaction mixture.

As for the intermittent provision of UV light, this feature allows for control over the reaction kinetics, and allows, if desired, to perform reaction in a sequential manner.

The oxygen provision periods may be of equal length, or may be of different length than the non-provision periods.

In case of more than one 1 oxygen provision period, the subsequent oxygen provision periods may be of equal length or may be of different length.

In case of more than one 1 non-provision period, the subsequent non-provision periods may be of equal length or may be of different length.

The provision of molecular oxygen into the reaction mixture can be carried out by using open reaction vessels that are exposed to ambient air. The provision of oxygen is preferably carried out by bubbling oxygen or air into the reaction mixture, wherein the above mentioned non-provision of oxygen can be achieved for example by bubbling an inert gas into the reaction mixture in place of the oxygen or air.

Alternatively, and in another embodiment of the Invention, the molecular oxygen is provided by the presence of a catalase enzyme in the reaction mixture, said catalase enzyme being able to convert hydrogen peroxide into molecular oxygen and water.

According to another preferred embodiment, the present invention concerns a process for chemical oxidation as described above,
wherein the step of exposing said at least one CRO enzyme to UV light is carried out before the step of contacting,
wherein said step of exposing to UV light before the step of contacting is maintained for 1 second to about 10 minutes, followed by contacting said UV-activated enzyme with said organic compound.

By “1 second to 10 minutes” should be understood all individual time-values comprised within this range, so that for example “1 second to 10 seconds” includes 1, 2, 3, 4, 5, 6, 7, 8, 9, and 10 seconds.

By “1 second to 10 minutes” should in particular be understood 1 second to 5 minutes, 1 second to 1 minute, 1 to 30 seconds, 1 to 10 seconds, 10 seconds to 10 minutes, 30 seconds to 10 minutes, 1 to 10 minutes, 5 to 10 minutes, 30 seconds to 5 minutes, 1 to 5 minutes, 2 to 5 minutes, or 2 to 3 minutes.

According to another preferred embodiment, the present invention concerns a process for chemical oxidation as described above,
wherein said process comprises between the step exposing said at least one CRO enzyme to UV light before the step of contacting, and the step of contacting said UV-activated enzyme with said organic compound,
a step of transfer whereby the organic compound and the UV-activated enzyme are mixed,
said step of transfer in particular being achieved in less than 10 minutes, preferably less than 1 minutes, and more preferably in less than 10 seconds.

With “a step of transfer” is meant the act of transferring the UV-activated enzyme into a solution comprising the organic substrate, or conversely transferring the organic substrate into the solution comprising the enzyme.

The transfer step is in particular carried out by transferring the UV-activated enzyme to the solution comprising the organic compound.

This transfer step should be performed quickly, preferably in less than 10 seconds, to prevent the UV-activated enzyme from deactivating in time.

In particular, the step of transfer should take place in a time sufficiently short to retain at least 80% of the initial activity of the UV-activated enzyme, preferably at least 90%, more preferably at least 95%.

In this respect the activity of the UV-activated enzyme is a parameter related to a batch of enzymes. The enzyme activity can be measured by methods known to the person skilled in the art.

According to another preferred embodiment, the present invention concerns a process for chemical oxidation as described above,
wherein said process is carried out in an aqueous medium, preferably in a buffered aqueous medium, more preferably at a temperature comprised between 20 and 50° C. preferably at a temperature of about 23° C.

The aqueous buffer is in particular a sodium phosphate buffer, citrate-phosphate buffer, a tris-HCl buffer or HEPES, preferably a sodium phosphate buffer.

The buffered aqueous medium preferably has a pH ranging from 6 to 10, in particular from 7 to 9, more in particular 7 or 8.

According to another preferred embodiment, the present invention concerns a process for chemical oxidation as described above,
wherein the at least one CRO enzyme is of fungal origin.

According to another preferred embodiment, the present invention concerns a process for chemical oxidation as described above,
wherein the at least one CRO enzyme is of bacterial origin.

According to another preferred embodiment, the present invention concerns a process for chemical oxidation as described above,
wherein the at least one CRO enzyme belongs to the AA5 family

Families and subfamilies of the enzymes used in the present enzymes are classified according to the CAZy database, which describes the families of structurally-related catalytic and carbohydrate-binding modules (or functional domains) of enzymes that degrade, modify, or create glycosidic bonds.

Alternatively, enzymes can be identified by an enzyme commission number, or EC-number:

| Enzyme | EC number |
|---|---|
| Galactose oxidase | 1.1.3.9 |
| Glyoxal oxidase | 1.2.3.15 |
| Alcohol oxidase | 1.1.3.13 |
| Aryl alcohol oxidase | 1.1.3.7 |

The at least one CRO enzyme in particular belongs to the AA5_1 or AA5_2 subfamilies.

According to another preferred embodiment, the present invention concerns a process for chemical oxidation as described above,
wherein the at least one CRO enzyme belongs to the AA5_2 subfamily and is an alcohol oxidase (AlcOx), preferably is an alcohol oxidase extracted from Colletotrichum graminicola, in particular having SEQ ID NO: 1, or having at least 60%, in particular at least 70% identity with SEQ ID NO: 1.

SEQ ID NO:1 corresponds to the following sequence, wherein the signal peptide is highlighted in underlined, and the catalytic domain is highlighted in bold:

MPTLRSALRNLPAALLALAAACEAQNVGKWGPMVK
FPVVPVAVALVPETGNLLVWSSGWPNRWTTAGNGK
TYTSLYNVNTGNISDAIVQNTQHDMFCPGTSLDAD
GRIIVTGGSSAAKTSVLDFKKGESSPWTPLSNMQI
SRGYQSSCTTSEGKIFVIGGSFSGAGTRNGEVYDP
KANTWTKLAGCPVKPLVMQRGMFPDSHAWLWSWKN
GSVLQAGPSKKMNWYDTKGTGSNTPAGLRGTDEDS
MCGVSVMYDAVAGKIFTYGGGKGYTGYDSTSNAHI
LTLGEPGQAVQVQKLANGKYNRGFANAVVMPDGKI
WVVGGMQKMWLFSDTTPQLTPELFDPATGSFTPTT
PHTVPRNYHSTALLMADATIWSGGGGLCGANCKEN
HFDGQFWSPPYLFEADGVTPAKRPVIQSLSDTAVR
AGAPITITMQDAGAYTFSMIRVSATTHTVNTDQRR
IPLDGQDGGDGKSFTVNVPNDYGVAIPGYYMLFAM
NEAGVPCVAQFFKVTL

According to the present Invention, with the expression “at least 70% identity” should also be understood: at least 71%, at least 72%, at least 73%, at least 74%, at least 75%, at least 76%, at least 77%, at least 78%, at least 79%, at least 80%at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

According to another preferred embodiment, the present invention concerns a process for chemical oxidation as described above,
wherein the at least one CRO enzyme belongs to the AA5_2 subfamily and is a galactose oxidase (GalOx) and more preferably is a galactose oxidase extracted from Fusarium graminearum, in particular having SEQ ID NO: 2, or having at least 60%, in particular at least 70% identity with SEQ ID NO: 2.

SEQ ID NO:2 corresponds to the following sequence, wherein the signal peptide is highlighted in underlined, and the catalytic domain is highlighted in bold:

MKHLLTLALCFSSINAVAVTVPHKAVGTGIPEGSL
QFLSLRASAPIGSAISRNNWAVTCDSAQSGNECNK
AIDGNKDTFWHTFYGANGDPKPPHTYTIDMKTTQN
VNGLSMLPRQDGNQNGWIGRHEVYLSSDGTNWGSP
VASGSWFADSTTKYSNFETRPARYVRLVAITEANG
QPWTSIAEINVFQASSYTAPQPGLGRWGPTIDLPI
VPAAAAIEPTSGRVLMWSSYRNDAFGGSPGGITLT
SSWDPSTGIVSDRTVTVTKHDMFCPGISMDGNGQI
VVTGGNDAKKTSLYDSSSDSWIPGPDMQVARGYQS
SATMSDGRVFTIGGSWSGGVFEKNGEVYSPSSKTW
TSLPNAKVNPMLTADKQGLYRSDNHAWLFGWKKGS
VFQAGPSTAMNWYYTSGSGDVKSAGKRQSNRGVAP
DAMCGNAVMYDAVKGKILTFGGSPDYQDSDATTNA
HIITLGEPGTSPNTVFASNGLYFARTFHTSVVLPD
GSTFITGGQRRGIPFEDSTPVFTPEIYVPEQDTFY
KQNPNSIVRVYHSISLLLPDGRVFNGGGGLCGDCT
TNHFDAQIFTPNYLYNSNGNLATRPKITRTSTQSV
KVGGRITISTDSSISKASLIRYGTATHTVNTDQRR
IPLTLTNNGGNSYSFQVPSDSGVALPGYWMLFVMN
SAGVPSVASTIRVTQ

According to another preferred embodiment, the present invention concerns a process for chemical oxidation as described above,
wherein the at least one CRO enzyme belongs to the AA5_2 subfamily and is an aryl alcohol oxidase (AAO) and is preferably extracted from Colletotrichum graminicola, in particular having SEQ ID NO: 3, or having at least 60%, in particular at least 70% identity with 20 SEQ ID NO: 3.

SEQ ID NO:3 corresponds to the following sequence, wherein the signal peptide is highlighted in underlined, and the catalytic domain is highlighted in bold:

MVRSCAYKAIAAASLLSQLASAAITSCPNNETVWE
TPIGVKYTLCPGSDYQNGGASLQTVRDIQSSLECA
KICDSDARCNRAVYDNVNKACDVKNSTNPMQWAAD
DRFETIRLTNDLPEGAFISTCSFNETSYRVPETNA
EYRICPDTDYTGVNAKVVEGVTTIQACAELCSNTQ
DCRKSVFDHINNACAIKAAEPATSIFWVQDKQFST
IRLPENIDPAVKGKWGDLIRLPVIPVAAYIVPSYP
EPSRLLFFSSWSNDAFSGASGMTQFGDYDFATGAI
SQRTVTNTHHDMFCPGISQLEDGRILIQGGSDADT
VSIYDPATNEFTRGPNMTLARGYQTSCTLSNGKVF
TIGGAYSGERVGKNGEVYDPVANAWTYLPGADFRP
MLTNDHEGIWREDNHAWLFGWKNGSIFQAGPSKDQ
HWYGIQGNGTVAKAATRDDDDAMCGVWVMYDAVAG
KIFSAGGSPDYTDSPATQRAHITTIGEPNTPAEVE
RVADMGFPRGFANAVVLPDGQVLVTGGQRMSLVFT
NTDGILVAELFNPETREWKQMAPMAVPRNYHSVSI
LLPDATVFSGGGGMCWVQNVGDSTAGCDKTVDHSD
GEIFEPPYLFNEDGSRAARPVISAISADPIKAGAT
LTFTVEGVEGQGTAALIRLGSVTHSVNSDQRRVPL
NVTVSGNEYSATLPDDYGILLPGYYYLFVSTPQGT
PSIAKTVHVIL

According to another preferred embodiment, the present invention concerns a process for chemical oxidation as described above,
wherein the at least one CRO enzyme belongs to the AA5_1 subfamily and is a glyoxal oxidase (GLOx) and more preferably is a glyoxal oxidase extracted from Pycnoporus cinnabarinus, in particular having SEQ ID NO: 4, or having at least 60%, in particular at least 70% identity with SEQ ID NO: 4.

SEQ ID NO:4 corresponds to the following sequence, wherein the signal peptide is highlighted in underlined, and the catalytic domain is highlighted in bold:

MFQTTLHLLFVLVVTGRGLAAPSTPTGWQFNLKAE
RSGIVALESIVVSPTLVVFFDRATNDPLQINNHSA
WGALWNLETSTVRALDVLTNSFCASGALLSNGTMA
SIGGDPNGFPGNPAIHPGTQAIRLFEPCDSPTGEG
CTLFEDPVTLHLLEKRWYPSSVRIFDGSLLIVGGM
HEETPFYNTDPALSFEFFPPKESTPRPSEFLNRSL
PANLFPRVFALPDGKVFMVANNQSIIYDIEANTER
ILPDIPNNVRVTNPIDGSAILLPLSPPDFVPEVLV
CGGTQTDTIDPSLLTSQTPASSQCSRIRLDEEGIA
RGWEVEHMLEGRMMPELVHLPNGQVLIANGARTGF
AAIASVSDPVGGSNADHAVLVPSLYTPDAPLGTRI
SNVGLPSSGIARVYHSSITLTPQGNFLIAGSNPNN
NSSVTAGVKFPSEFRVQTLDPPFMFVERPKILSMP
KKLAFGKSFTVPIAVPSTLAHPGAKVQVSLMDLGF
SSHAFHSSARLVFMNAKISQDGKSLTFTTPPNGRV
YPPGPATIFLTIDDVTSEGAWVMMGSGNPPPTLE

According to another preferred embodiment, the present invention concerns a process for chemical oxidation as described above,
wherein the at least one CRO enzyme is a GlxA-type enzyme which is preferably extracted from the bacterium Streptomyces lividans, in particular having SEQ ID NO: 5, or having at least 60%, in particular at least 70% identity with SEQ ID NO: 5.

GlxA enzymes are bacterial homologues of fungal CAZy family AAS, in particular of galactose oxidases.

SEQ ID NO:5 corresponds to the following sequence, wherein the signal peptide is highlighted in underlined, and the catalytic domain is highlighted in bold:

MKDRAGRRRARRFAIGTAVVVALAGMNGPWLYRFS
TEKYHQYKINQPEYKAANGKWEIIEFPEKYRQNTI
HAALLRTGKVLMVAGSGNNQDNSDDKQYDTRIWDP
VKGTIKKVPTPSDLFCTGHTQLANGNLLIAGGTKR
YEKLKGDVTKAGGLMVVHNENPDKPITLPAGTKFT
GKENGKTFVSKDPVLVPRAEKVFDPATGAFVRNDP
GLGRIYVEAQKSGSAYETGTEDNYRVQGLSGADAR
NTYGIAQKLALDKKDFQGIRDAFEFDPVAEKYIKV
DPMHEARWYPTLTTLGDGKLSVSGLDDIGQLVPGK
NEVYDPKTKAWTYTDKVRQFPTYPALFLMQNGKIF
YSGANAGYGPDDVGRTPGVWDVETNKFTKVPGMSD
ANMLETANTVLLPPAQDEKYMVIGGGGVGESKLSS
EKTRIADLKADDPKFVDGPSLEKGTRYPQASILPD
DSVLVSGGSQDYRGRGDSNILQARLYHPDTNEFER
VADPLVGRNYHSGSILLPDGRLMFFGSDSLYADKA
NTKPGKFEQRIEIYTPPYLYRDSRPDLSGGPQTIA
RGGSGTFTSRAASTVKKVRLIRPSASTHVTDVDQR
SIALDFKADGDKLTVTVPSGKNLVQSGWYMMFVTD
GEGTPSKAEWVRVP

According to another preferred embodiment, the present invention concerns a process for chemical oxidation as described above,
wherein the at least one CRO enzyme belongs to the AA5_2 subfamily and is an alcohol oxidase (AlcOx), preferably is an alcohol oxidase extracted from Colletotrichum graminicola, in particular having SEQ ID NO: 1, or having at least 60%, in particular at least 70% identity with SEQ ID NO: 1,
or wherein the at least one CRO enzyme belongs to the AA5_2 subfamily and is a galactose oxidase (GalOx) and more preferably is a galactose oxidase extracted from Fusarium graminearum, in particular having SEQ ID NO: 2, or having at least 60%, in particular at least 70% identity with SEQ ID NO: 2.
or wherein the at least one CRO enzyme belongs to the AA5_2 subfamily and is an aryl alcohol oxidase (AAO) and is preferably extracted from Colletotrichum graminicola, in particular having SEQ ID NO: 3, or having at least 60%, in particular at least 70% identity with SEQ ID NO: 3.
or wherein the at least one CRO enzyme belongs to the AA5_1 subfamily and is a glyoxal oxidase (GLOx) and more preferably is a glyoxal oxidase extracted from Pycnoporus cinnabarinus, in particular having SEQ ID NO: 4, or having at least 60%, in particular at least 70% identity with SEQ ID NO: 4.
or wherein the at least one CRO enzyme is a GlxA-type enzyme which is preferably extracted from the bacterium Streptomyces lividans, in particular having SEQ ID NO: 5, or having at least 60%, in particular at least 70% identity with SEQ ID NO: 5.

According to another preferred embodiment, the present invention concerns a process for chemical oxidation as described above, wherein said organic compound is chosen from:
saturated (C₁ to C₂₀) primary alcohols,
unsaturated (C₁ to C₂₀) primary alcohols,
saturated (C₁ to C₂₀) secondary alcohols,
unsaturated (C₁ to C₂₀) secondary alcohols,
(C₃ to C₁₀) cyclic alcohols,
aryl alcohols,
heteroaryl alcohols, and
geminal diols.

According to another preferred embodiment, the present invention concerns a process for chemical oxidation as described above, wherein said organic compound is chosen from:
saturated (C₁ to C₂₀) primary alcohols,
unsaturated (C₁ to C₂₀) primary alcohols,
aryl alcohols comprising a primary hydroxyl group,
heteroaryl alcohols comprising a primary hydroxyl group, and
geminal diols.

According to another preferred embodiment, the present invention concerns a process for chemical oxidation as described above, wherein said organic compound is chosen from:
saturated (C₁ to C₂₀) primary alcohols,
allylic alcohols,
aryl alcohols comprising a primary hydroxyl group linked to the aryl group by a C₁ alkyl group, and
geminal diols.

The alcohols according to the present invention can also comprise more than one hydroxyl groups. Examples of such polyols are 1,2-propanediol, 1,3-propanediol, 1,4-propanediol, glycerol, sorbitol, xylitol,
or carbohydrates, in particular carbohydrates chosen from the group consisting of sugars such as galactose, lactose, melibiose, raffinose, glucose, xylose, arabinose, ribose, fructose, mannose, sucrose, lactose, cellobiose and xyloglucan,
or macromolecules, in particular natural polymers comprising aliphatic chains bearing hydroxyl groups, such plant cutins.

According to the present invention, primary alcohols are oxidized into aldehydes, secondary alcohols are oxidized into ketones, and gem-diols are oxidized into carboxylic acids.

The invention in particular concerns primary alcohols being oxidized into aldehydes and gem-diols being oxidized into carboxylic acids.

The term “saturated (C1 to C20) primary alcohols” means a primary alcohol comprising a saturated alkyl chain comprising 1 to 20 carbon atoms, linear or branched, in particular comprising 1, 2, 3, 4, 5, 6, 7, 8, 9, 10, 11, 12, 13, 14, 15, 16, 17, 18, 19, or 20 carbon atoms, said saturated primary alcohols being for example chosen from methanol, ethanol, 1-propanol, 1-butanol, 1-pentanol, 1-hexanol, 1-heptanol, (±)-2-methyl-1-butanol and (S)-(−)-2-methyl-1-butanol,
in particular methanol being oxidized to formaldehyde, or 1-hexanol being oxidized to 1-hexanal.

The term “unsaturated (C1 to C20) primary alcohols” means a primary alcohol comprising an alkyl chain comprising 1 to 20 carbon atoms, linear or branched, in particular comprising 1, 2, 15 3, 4, 5, 6, 7, 8, 9, 10, 11, 12, 13, 14, 15, 16, 17, 18, 19, or 20 carbon atoms, said alkyl chain further comprising one or more double, or triple bonds,
in particular allylic alcohols,
said unsaturated primary alcohols being for example chosen from cis-3-hexen-1-ol, 2,4-hexadiene-1-ol, retinol and geraniol.

The term “saturated (C1 to C20) secondary alcohols” means a secondary alcohol comprising a saturated alkyl chain comprising 1 to 20 carbon atoms, linear or branched, in particular comprising 1, 2, 3, 4, 5, 6, 7, 8, 9, 10, 11, 12, 13, 14, 15, 16, 17, 18, 19, or 20 carbon atoms, said saturated secondary alcohols being for example chosen from 2-propanol, 2-butanol.

The term “unsaturated (C1 to C20) secondary alcohols” means a secondary alcohol comprising an alkyl chain comprising 1 to 20 carbon atoms, linear or branched, in particular comprising 1, 2, 3, 4, 5, 6, 7, 8, 9, 10, 11, 12, 13, 14, 15, 16, 17, 18, 19, or 20 carbon atoms, said alkyl chain further comprising one or more double, or triple bonds,

The term “(C₃ to C₁₀) cyclic alcohols” means a cyclic compound comprising from 3 to 10 carbon atoms, in particular comprising 3, 4, 5, 6, 7, 8, 9 or 10 carbon atoms,
said cycle can be saturated or unsaturated,
said cycle can further comprise a heteroatom, in particular chosen from O, N, or S,
said cycle comprising at least one hydroxyl group, said cyclic alcohols being for example chosen from cyclopentanol; 2-cyclopentenol, cyclohexanol and 2-cyclohexenol,

The term “aryl alcohols” means a primary or secondary alcohol further comprising at least one aryl group, said at least one aryl group being linked to the hydroxyl group, in particular a primary hydroxyl group, by a (C₁ to C₂₀) linear or branched alkyl chain as previously defined, in particular by a C₁ alkyl chain,
said aryl alcohols being in particular aryl alcohols comprising a primary hydroxyl group linked to the aryl group by a C₁ alkyl group, or aryl alcohols comprising an allylic alcohol attached to the aryl group,
said aryl alcohols being for example chosen from benzyl alcohol, 4-methoxy benzyl alcohol, 2,3-dimethoxy benzyl alcohol, 4-hydroxy benzyl alcohol, 2-phenyl ethanol, cinnamyl alcohol, coniferyl alcohol, sinapyl alcohol,
in particular benzyl alcohol being oxidized to benzaldehyde.

The term “heteroaryl alcohols” means an aryl alcohol as previously defined, wherein the aryl group further comprised a heteroatom chosen from O, N, or S,
said heteroaryl alcohols being for example chosen from furfuryl alcohol and p-coumaryl alcohol.

The term “geminal diols” means a compound of structure R₁HC(OH)₂, being oxidized into R₁CO₂H· wherein R1 represents
a (C₁ to C₂₀) alkyl group, linear or branched,
a (C3 to C₁₀) cyclic alkyl group,
a carboxylic acid group,
an aryl group, or heteroaryl.

In another embodiment, the present invention concerns a process for chemical oxidation as described above, wherein said organic compound is of natural origin, or is derived from a compound of natural origin.

Examples of such compounds are waxes, cutins, hemicellulose such as xyloglucan,

According to another preferred embodiment, the present invention concerns a process for chemical oxidation as described above, wherein said organic compound is chosen from:
saturated (C₁ to C₂₀) primary alcohols,
unsaturated (C₁ to C₂₀) primary alcohols,
saturated (C₁ to C₂₀) secondary alcohols,
unsaturated (C₁ to C₂₀) secondary alcohols,
(C3 to C₁₀) cyclic alcohols,
aryl alcohols,
heteroaryl alcohols, and
geminal diols,
in particular chosen from:
   saturated (C₁ to C₂₀) primary alcohols,
   allylic alcohols,
   aryl alcohols comprising a primary hydroxyl group linked to the aryl group by a C₁ alkyl group, and
   geminal diols.

According to another preferred embodiment, the present invention concerns a process for chemical oxidation as described above,
wherein said enzyme is an alcohol oxidase (AlcOx), and wherein said organic compound is a primary alcohol, or an aryl alcohol, and the obtained oxidized organic product is an aldehyde.

According to another preferred embodiment, the present invention concerns a process for chemical oxidation as described above, wherein said enzyme is an alcohol oxidase (AlcOx), and wherein said organic compound is an aryl alcohol comprising a primary hydroxyl group linked to the aryl group by a C₁ alkyl group, in particular selected from benzyl alcohol, 4-nitrobenzyl alcohol, anisyl alcohol, veratryl alcohol and 4-hydroxybenzyl alcohol,
or aryl alcohols comprising an allylic alcohol attached to the aryl group, in particular cinnamyl alcohol.

According to another preferred embodiment, the present invention concerns a process for chemical oxidation as described above, wherein said enzyme is an alcohol oxidase (AlcOx), and wherein said organic compound is a saturated, or unsaturated (C₁ to C₂₀) primary alcohol, linear or branched, in particular chosen from n-butanol, n-pentanol, n-hexanol or 2,4-hexadiene-1-ol.

According to another preferred embodiment, the present invention concerns a process for chemical oxidation as described above, wherein said enzyme is an alcohol oxidase (AlcOx), and wherein said organic compound is a naturally-occurring polymer comprising long aliphatic chains bearing hydroxyl functions or a sugar, in particular polymers chosen from waxes, cutins and hemicellulose.

According to another preferred embodiment, the present invention concerns a process for chemical oxidation as described above, wherein said enzyme is an alcohol oxidase (AlcOx), and wherein said organic compound is:
a primary alcohol and the obtained oxidized organic product is an aldehyde,
an aryl alcohol comprising a primary hydroxyl group linked to the aryl group by a C1 alkyl group, in particular selected from benzyl alcohol, 4-nitrobenzyl alcohol, anisyl alcohol, veratryl alcohol and 4-hydroxybenzyl alcohol, or aryl alcohols comprising an allylic alcohol attached to the aryl group, in particular cinnamyl alcohol,
a saturated, or unsaturated (C1 to C20) primary alcohol, linear or branched, in particular chosen from n-butanol, n-pentanol, n-hexanol or 2,4-hexadiene-1-ol, or
a naturally-occurring polymer comprising long aliphatic chains bearing hydroxyl functions or a sugar, in particular polymers chosen from waxes, cutins and hemicellulose.

According to another preferred embodiment, the present invention concerns a process for chemical oxidation as described above, wherein said enzyme is a glyoxal oxidase (GLOx), and wherein said organic compound is:
5-hydroxymethylfurfuryl alcohol, or
lignocellulose derived compounds, in particular glyoxal, methyl glyoxal, glyoxylic acid, formaldehyde or glycerol.

Said glyoxal, methyl glyoxal, glyoxylic acid and formaldehyde being in particular in the form of a hemi-acetal.

According to another preferred embodiment, the present invention concerns a process for chemical oxidation as described above,
wherein said enzyme is a galactose oxidase (GalOx), and wherein said organic compound is:
   forest and agricultural biomass, in particular fibres, and hemicelluloses, in particular compounds comprising a galactopyranose moiety, more in particular xyloglucan.

A third object of the present invention is a process for the production of hydrogen peroxide, said process comprising the step of contacting one organic compound bearing an oxidizable function with at least one Copper-Radical Oxidase (CRO) enzyme in the presence of molecular oxygen, under exposure to UV light,
whereby hydrogen peroxide is generated.

A fourth object of the present invention is the use of the hydrogen peroxide obtained by a process for the production of hydrogen peroxide as previously described, or by a process for chemical oxidation as described above,
in hydrogen peroxide-driven enzymatic reactions.

According to another preferred embodiment, the present invention concerns the use of hydrogen peroxide as previously described, wherein the hydrogen peroxide-driven enzymatic reaction is chosen from decarboxylations, hydroxylations, halogenations, epoxidations, sulfoxidations, Baeyer-Villiger oxidations.

According to another preferred embodiment, the present invention concerns the use of hydrogen peroxide as previously described, wherein the hydrogen peroxide-driven enzymatic reaction consists in the enzymatic conversion of said hydrogen peroxide into oxygen and water, in particular using a catalase enzyme.

In this embodiment, the hydrogen peroxide that is formed from molecular oxygen during the CRO-catalyzed oxidation reaction is re-converted into molecular oxygen. Thus, regeneration of molecular oxygen occurs, allowing for the CRO-catalyzed oxidation reaction to continue to take place.

According to another preferred embodiment, the present invention concerns the use of hydrogen peroxide as previously described, wherein the hydrogen peroxide-driven enzymatic reactions consists in the degradation of a polysaccharide, said reaction comprising contacting said polysaccharide with one or more lytic polysaccharide monooxygenase (LPMO), in the presence of an external source of electrons,
said source of electrons being in particular a reducing agent.

Examples of reducing agents are organic compounds such as ascorbic acid or cysteine, photocatalysts such as Titanium dioxide, or enzymes such as cellobiose dehydrogenase.

According to another preferred embodiment, the present invention concerns the use of hydrogen peroxide as previously described,
wherein the hydrogen peroxide-driven enzymatic reaction is chosen from decarboxylations, hydroxylations, halogenations, epoxidations, sulfoxidations and B aeyer—Villiger oxidations,
or wherein the hydrogen peroxide-driven enzymatic reaction consists in the enzymatic conversion of said hydrogen peroxide into oxygen and water, in particular using a catalase enzyme,
or wherein the hydrogen peroxide-driven enzymatic reactions consists in the degradation of a polysaccharide, said reaction comprising contacting said polysaccharide with one or more lytic polysaccharide monooxygenase (LPMO), in the presence of an external source of electrons, said source of electrons being in particular a reducing agent.

The following figures and examples illustrate the practice of the present invention in some of its embodiments, the figures and examples should not be construed as limiting the scope of the invention.

### FIGURES

FIG. 1 represents the EPR spectra showing the UV-activation of a CRO enzyme. FIG. 1A represents a non-activated AA5_2 AlcOx enzyme, and FIG. 1B the enzyme after UV activation. The arrows identify signals that appear after the UV-activation.

FIG. 2 represents the conversion of benzyl alcohol into benzaldehyde by an AlcOx CRO under different reaction conditions. The y-axis corresponds to the benzaldehyde concentration (μM) and the x-axis corresponds to the reaction time expressed in minutes.

▪ represents a reaction without activation of the enzyme (control reaction), ▴ represents a reaction wherein the enzyme is activated with horseradish peroxidase, ● represents a reaction according to the Invention, the enzyme being pre-activated with UV light, and ♦ represents a reaction wherein the buffer is exposed to UV light, in the absence of enzyme (negative control)

FIG. 3 represents the conversion of benzyl alcohol into benzaldehyde by an AlcOx CRO under different reaction conditions. The y-axis corresponds to the benzaldehyde concentration (μM) and the x-axis corresponds to the reaction time expressed in minutes.

▪ represents a reaction in the dark, without UV light (control reaction), ▴ represents a reaction with intermittent UV exposure, but in the absence of a CRO enzyme (negative control), ● represents a reaction according to the Invention, wherein the enzyme was pre-activated by UV, and ♦ represents a reaction according to the Invention, wherein the enzyme intermittently activated by UV light, the grey vertical bars represent the periods of light exposure.

FIG. 4 represents the light intensity-dependent activity of CgrAlcOx as determined according to the experiment of example 13. The y-axis corresponds to the initial rate Vᵢ/[E] of benzyl alcohol oxidation to benzaldehyde, expressed in s⁻¹, and the x-axis corresponds to the reaction light intensity (%). The reaction mixtures having been exposed to varying intensities of either UV light (λ=280±10 nm) or broad spectrum UV-Vis light (λ=200-800 nm). Error bars show standard deviation (n=3, independent experiments)

□ represents a reactions using UV light.

⋄ represents a reaction using broad spectrum UV-Vis light.

FIG. 5 represents the verification of linearity between the light intensity set on the apparatus and the measured photon flux at 4 cm from optic fiber outlet. Experiments were carried out with UV light (λ=280±10 nm). The photon flux was measured with a photometer calibrated at 280 nm. The y-axis shows the measured photon flux at 4 cm, expressed in mW/cm², the x-axis shows the light intensity of the light source, in %. Error bars show standard deviation (n=10, independent experiments)

FIG. 6 shows the impact of the distance between optic fiber outlet and the sample on the light intensity received by the latter. Experiments were carried out at 40% of Iₘₐₓ (Iₘₐₓ (at 4 cm, λ=280±10 nm)=1.6 mW·cm⁻², 36.5 μmol photon·s⁻¹·m⁻²). The y-axis shows the measured photon flux, expressed in mW/cm², the x-axis shows the distance from the optic fiber outlet, in cm. Error bars show standard deviation (n=10, independent experiments).

FIG. 7 shows the effect of duration of light exposure on CgrAlcOx activity as determined according to the experiment of example 14. The y-axis corresponds to the benzaldehyde concentration, expressed in mM, and the x-axis corresponds to the reaction time (min), of being exposed to varying illumination modes: continuous UV light (λ=280±10 nm), discontinuous UV light (λ=280±10 nm), discontinuous broad spectrum UV-Vis light (200-800 nm), or not having been exposed (dark). Error bars show standard deviation (n=3, independent experiments).

x represents continuous UV light.

□ represents discontinuous UV light.

⋄ represents discontinuous broad spectrum UV-Vis light.

● represents non light exposure (dark).

FIG. 8 represents the effect of pre-photoactivation on CgrAlcOx activity as determined according to the experiment of example 15. The y-axis corresponds to the initial rate Vᵢ/[E] of benzyl alcohol oxidation to benzaldehyde, expressed in s⁻¹, and the x-axis corresponds to the reaction time (seconds). Error bars show standard deviation (n=3, independent experiments).

FIG. 9 represents the effect of discontinuous illumination mode on CgrAlcOx activity, as determined according to the experiment of example 16. The y-axis corresponds to the benzaldehyde concentration, expressed in mM, and the x-axis corresponds to the reaction time (min). The reaction mixtures having been exposed to UV light with two different discontinuous illumination modes: on/off cycles of either 2/30 min or 10/30 min. Error bars show standard deviation (n=3, independent experiments).

□ represents on/off cycles of 2/30 min

⋄ represents on/off cycles of 10/30 min.

FIG. 10 represents the Photo-activation of CgrAAO, as determined according to the experiment of example 17. FIG. 10(A) corresponds to the results obtained for benzyl alcohol oxidation. FIG. 10(B) corresponds to the results obtained for 5-hydroxymethylfurfural oxidation. FIG. 10(C) corresponds to the results obtained for 5-hydroxymethyl-2-furan carboxylic acid oxidation

The error bars show the standard deviation for 2 independent experiments.

The y-axes correspond to the aldehyde product concentrations, expressed in mM, and the x-axes correspond to the reaction time (min).

□ represents absence of activation (reaction in the dark).

⋄ represents activation by discontinuous UV light.

FIG. 11 represents the synergy between CgrAlcOx and catalase as determined according to the experiment of example 18. The y-axis corresponds to the concentration of benzaldehyde, expressed in mM, and the x-axis corresponds to the reaction time (min), having been exposed to discontinuous UV light (λ=280±10 nm), in the absence or presence of catalase (5 nM final). Error bars show standard deviation (n=3, independent experiments).

Δ represents the absence of catalase.

x represents the presence of catalase.

FIG. 12 represents the stability of benzaldehyde (▪) and benzyl alcohol (x) under light exposure in the absence of enzyme, as determined according to the experiment of example 19. The y-axis corresponds to the concentration of benzaldehyde, expressed in mM, and the x-axis corresponds to the reaction time (min).

FIG. 13 represents the photoactivation on FgrGalOx activity as determined according to the experiment of example 20.

FIG. 13(A) corresponds to the results obtained for benzyl alcohol oxidation, in which the y-axis corresponds to the concentration of benzaldehyde, expressed in mM, and the x-axis corresponds to the reaction time (min). Error bars show standard deviation (n=3, independent experiments).

Δrepresents absence of activation (reaction in the dark).

⋄ represents activation by UV light.

FIG. 13(B) corresponds to the results obtained for lactose oxidation, in which the y-axis corresponds to the concentration of oxidized lactose, expressed in mM, and the x-axis corresponds to the illumination time (min). Error bars show standard deviation (n=3, independent experiments).

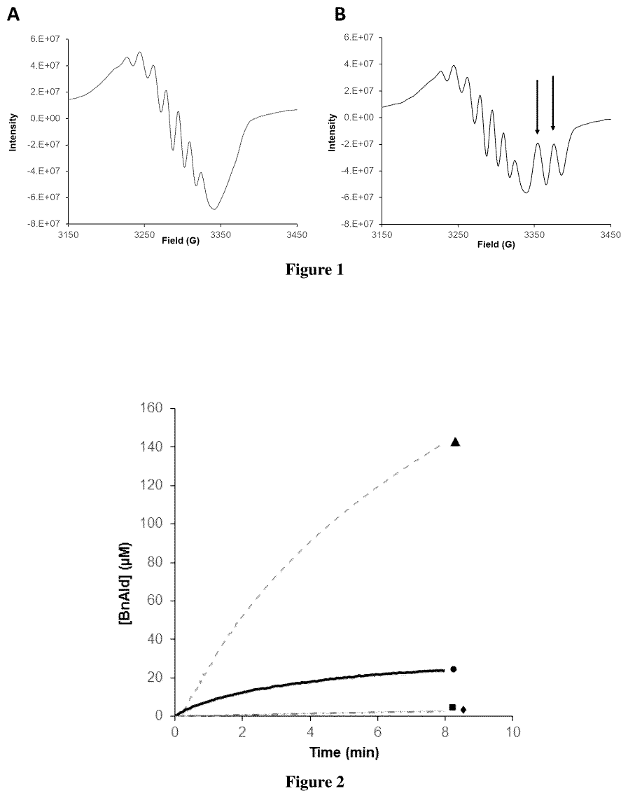

### EXAMPLES

Abbreviations and definitions

| | |
|---|---|
| BMGY medium | Buffered Glycerol-complex Medium |
| BMMY medium | Buffered Methanol-complex. Medium |
| EPR | Electron Paramagnetic Resonance |
| HRP | Horseradish peroxidase |
| OD_{600 nm} | Optical Density measured at 600 nm |
| Pluronic E8100 | Non-ionic surfactant (anti-foam) |
| PTM1 trace salts | Salt-metals mix solution |
| Rpm | Revolutions per minute |
| YPD agar | Yeast Extract Peptone Dextrose Agar |
| g | relative centrifugal force |

### General Remarks

Most chemicals were purchased from Sigma-Aldrich (Darmstadt, Germany) or VWR. HRP type II (MW 33.89 kDa) and catalase from bovine liver (monomer MW 62.5 kDa) were purchased from Sigma-Aldrich.

Molar concentrations of HRP was estimated by Bradford assay.

All alcohol substrates stock solutions were prepared in H₂O whenever possible, aliquoted and stored at −20° C. The concentration of H₂O₂ stock solution was verified at 240 nm (ε²⁴⁰=43.6 M⁻¹·cm⁻¹).

### Example 1: DNA Cloning and strain production

The DNA cloning and strain production was performed according to methods described in the literature:
DNA cloning and strain production of the alcohol oxidase from Colletotrichum graminicola (CgrAlcOx, Genbank ID XM_008096275.1, Uniprot ID E3QHV8) was performed according to D. (Tyler) Yin et al., “Structure—function characterization reveals new catalytic diversity in the galactose oxidase and glyoxal oxidase family,” Nat. Commun., vol. 6, p. 10197, December 2015.
DNA cloning and strain production of the galactose oxidase from Fusarium graminearum (FgrGalOx, Genbank ID XM_011327027.1, Uniprot ID I1S2N3) was performed according to O. Spadiut et al., “A comparative summary of expression systems for the recombinant production of galactose oxidase,” Microb. Cell Fact., vol. 9, pp. 1-13, 2010.
DNA cloning and strain production of the glyoxal oxidase from Pycnoporus cinnabarinus (GLOx, PciGLOx-2, ORF ID BN946_scf184747.g42, Uniprot ID A0A060SYB0) was performed according to M. Daou et al., “Heterologous production and characterization of two glyoxal oxidases from Pycnoporus cinnabarinus,” Appl. Environ. Microbiol., vol. 82, no. 16, pp. 4867-4875, 2016.
DNA cloning and strain production of the aromatic alcohol oxidase from Colletotrichum graminicola (CgrAAO, Genbank ID EFQ27661, Uniprot ID E3Q9X3) was performed according to Y. Mathieu et al., “Discovery of a Fungal Copper Radical Oxidase with High Catalytic Efficiency toward 5-Hydroxymethylfurfural and Benzyl Alcohols for Bioprocessing,” ACS Catal., vol. 10, no. 5, pp. 3042-3058, 2020.

### Example 2: Heterologous Enzyme Production

For preliminary tests, all proteins were first produced in 2 L flasks. To this end, single colonies of P. pastoris X33 expressing the gene of interest were individually streaked on a YPD agar plate containing Zeocin (100 μg·mL⁻¹) and incubated 3 days at 30° C. A single colony was then used to inoculate 10 mL of YPD, in a 50 mL sterile Falcon tube, incubated during 5 h (30° C., 160 rpm). This pre-culture was used to inoculate at 0.2% (vol/vol), 500 mL of BMGY medium in a 2 L baffled flask, incubated during approximately 16 h (30° C., 200 rpm) until the OD_{600 nm} reached 4-6. The produced cellular biomass was then harvested by centrifugation (5 min, 16° C., 3,000 g). The cell pellet was resuspended in 100 mL BMMY medium in a 500 mL flask supplemented with CuSO₄ (500 μM) and methanol (1%, vol/vol) and incubated for 3 days (16° C., 200 rpm), with daily additions of methanol (1% added, vol/vol). Then, the extracellular medium was recovered by centrifugation (10 min, 4° C., 3,000 g) and the supernatant filtered on 0.45 μm membrane (Millipore, Massachusetts, USA) and stored at 4° C. prior to purification.

CgrAAO and PciGLOx-2 were produced according to known procedures: Y. Mathieu et al., “Discovery of a Fungal Copper Radical Oxidase with High Catalytic Efficiency toward 5-Hydroxymethylfurfural and Benzyl Alcohols for Bioprocessing,” ACS Catal., vol. 10, no. 5, pp. 3042-3058, 2020 and M. Daou et al., “Heterologous production and characterization of two glyoxal oxidases from Pycnoporus cinnabarinus,” Appl. Environ. Microbiol., vol. 82, no. 16, pp. 4867-4875, 2016, respectively.

### Example 3: Heterologous Enzyme Bioreactor Production

CgrAlcOx and FgrGalOx were also produced at larger scale, in bioreactors. Bioreactor production was carried out in a 1.3-L New Brunswick BioFlo 115 fermentor (Eppendorf, Germany) Precultures were prepared as described above for flask production and were used to inoculate at 0.2% 100 mL of BMGY medium, in a 500 mL flask, incubated (30° C., 200 rpm) until the OD_{600 nm} reached 2-6. Four hundred mL of basal salt medium containing 1.8 mL PTM1 trace salts (both made according to the P. pastoris fermentation process guidelines—Invitrogen—version B 053002) were inoculated with 10% (vol/vol) of the BMGY culture. Temperature was set to 30° C. One hundred μL of Pluronic E8100 (BASF, Germany) were added after 6 h of culture to prevent foam. After full consumption of glycerol (as indicated by a return of dissolve oxygen (DO) level at 100%), sorbitol-methanol transition phase was initiated by addition of 80 mL sorbitol (250 g·L⁻¹ stock solution), 1.6 mL PTM1 traces salts and 2 mL methanol. After full consumption of carbon sources, the temperature was lowered to 20° C. and a methanol fed-batch was initiated with a feeding rate of 3.9 mL/h/L (mL per hour per liter of initial fermentation volume) of a methanol solution complemented with PTM1 trace salts (12 mL/L). New additions of 100 μL Pluronic E8100 were made after 30 h and 53 h of fermentation. Methanol feeding rate was increased to 7.8 mL/h/L after 53 h of fermentation. Throughout the fermentation, pH was maintained at 5 by automated adjustment with NH₃ base. Air flow was maintained at 0.5 slpm (standard liter per minute). A cascade with a set point of 20% dissolved oxygen is maintained through agitation between 400 to 900 rpm and the percentage of pure oxygen addition between 0 to 50%. Fermentation was ceased after 118 hours. The harvested biomass was centrifuged (10 min, 5500 g, 4° C.). The supernatant was filtered through a 0.45-μm membrane (Millipore), flash-frozen in liquid nitrogen and stored at −80° C.

Flash-freezing did not cause any enzyme activity loss, for both CgrAlcOx and FgrGalOx.

### Example 4: Protein Purification

The filtered culture broth was buffer exchanged by ultrafiltration through a 10 kDa cut-off polyethersulfone membrane (Vivacell 250, Sartorius Stedim Biotech GmbH, Germany) with Tris-HCl(50 mM pH 8.7) or Tris-HCl (50 mM pH 8.0) for CRO-AlcOx and CRO-GalOx, respectively. CRO-AlcOx was purified by anion exchange chromatography by loading the crude protein sample on a DEAE-20 mL HiPrep FF 16/10 column (GE Healthcare, Illinois, USA), equilibrated with buffer A1 (Tris-HCl, 50 mM, pH 8.7) and connected to an Äkta Express system (GE Healthcare). Elution was performed by applying a linear gradient from 0 to 50% of buffer B1 (Tris-HCl, 50 mM, pH 8.7+1 M NaCl) over 15 column volumes (CV) at a flow rate of 3 mL·min⁻¹.

FgrGalOx was purified by ionic metal affinity chromatography by loading the crude protein sample on a His-Trap HP 5-mL column (GE Healthcare, Buc, France) and connected to an Äkta Xpress system (GE Healthcare). Prior to loading, the column was equilibrated with buffer A2 (50 mM Tris-HCl, pH 7.8, 150 mM NaCl). After loading, non-specific proteins were eluted by applying a first washing step of 5 CV at 2% of buffer B2 (50 mM Tris-HCl, pH 7.8, 150 mM NaCl, 500 mM imidazole) and the target protein was eluted during a second step of 5 CV at 30% of buffer B2. Loading and elution were carried out at a flow rate of 3 mL·min⁻¹. In all cases, the collected fractions were analyzed by SDS-PAGE in 10% polyacrylamide precast gel (Bio-Rad) stained with Coomassie blue. Fractions containing the recombinant enzyme were pooled, concentrated and buffer exchanged in sodium phosphate (50 mM, pH 7.0) or sodium acetate (50 mM, pH 5.2), for CgrAlcOx and FgrGalOx, respectively. The protein concentration was determined by UV absorption at 280 nm using a Nanodrop ND-200 spectrophotometer (Thermo Fisher Scientific, Massachusetts, USA) for CgrAlcOx (ε=101215 M⁻¹·cm⁻¹), FgrGalOx (ε=124,135 M⁻¹·cm⁻¹) , PciGLOx-1 (ε=47,690 M⁻¹·cm⁻¹), CgrAAO (ε=107,760 M⁻¹·cm⁻¹).

### Example 5: Spectrophotometric Monitoring of Benzyl Alcohol Oxidation

Benzyl alcohol can be oxidized by several CROs, including CgrAlcOx, FgrGalOx and CgrAAO. The oxidation of benzyl alcohol into benzaldehyde was monitored by developing a simple absorbance assay consisting in measuring changes in absorbance at 254 nm upon oxidation of benzyl alcohol (1.5 mM) by the CRO (10 nM final concentration). Reactions were carried out in sodium-phosphate buffer (50 mM, pH 7.0), at 23° C., in UV-transparent cuvettes (1 mL reaction volume). In positive controls (carried out in the dark), horseradish peroxidase (HRP, 50 μg mL⁻¹) was added to the mixture. The reactions were initiated by adding the CRO (100 μL of 100 nM stock solution), which was either stored in the dark or UV-exposed (vide infra). The reaction was mixed by vigorously pipetting up and down. In negative controls, the enzyme stock solution was replaced by sodium phosphate buffer (50 mM, pH 7.0), kept in the dark or UV-exposed. The absorbance (pathlength=1 cm) was measured using an Evolution 201 UV-Vis spectrophotometer (Thermo-Fisher). Given a 1:1 stoichiometry of the reaction, one can calculate the concentration of benzaldehyde according to Equation 1, where the molecular extinction coefficient of benzaldehyde at 254 nm is 57-fold higher (ε²⁵⁴_{benzaldehyde}=8,497 M⁻¹·cm⁻¹) than its alcohol counterpart (ε²⁵⁴_{BnoH=}149 M⁻¹·cm⁻¹).

[Benzaldehyde]ₜ=(Abs^{254 nm}ₜ−Abs^{254 nm}ₜ₀)/(ε²⁵⁴ _{benzaldehyde}−ε²⁵⁴_{BnOH}) (equation 1)

### Example 6: CRO Pre-Photoactivation

Pre-activation experiments consisted in exposing the enzyme stock solution (100 nM, 1 mL) to UV light during a given amount of time (usually 10 min) before transferring (ca. 30 sec step) a fraction (100 μL) to the reaction mixture. The reaction was then monitored as described above. In preliminary tests, a UV-lamp model EF-180/F (Spectroline, Spectronics Corporation, Westbury, USA) was used, delivering 254 nm UV light at a maximum power of 1350 μW/cm², equivalent to 29 μmoles of photon·s⁻¹·m⁻²), positioned side-wise (relatively to the reaction cuvette) at a 3 cm distance from the cuvette.

FIG. 2 shows the reaction kinetics observed for the benzaldehyde formation using an AlcOx CRO enzyme.

### Example 7: CRO Intermittent Illumination

In intermittent illumination experiments, the full reaction mixture, i.e. containing buffer, CRO and benzyl alcohol, was submitted to cycles of UV exposure (2 min) followed by on-line spectrophotometric monitoring (2 min). In such experiments, the illumination set-up was the same as described in example 6.

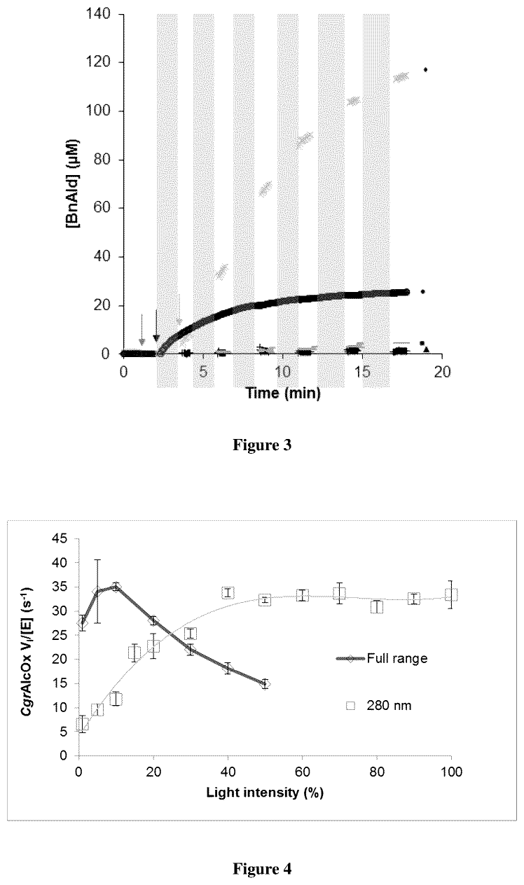

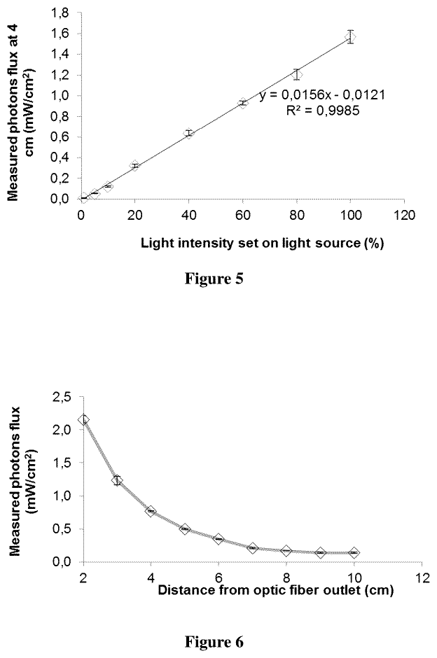

FIG. 3 shows the reaction kinetics observed for the benzaldehyde formation using an AlcOx CRO enzyme, an applying intermittent UV light exposure.

### Example 8: Electron Paramagnetic Resonance

CgrAlcOx (50 μM), prepared in sodium phosphate buffer (50 mM, pH 7.0), was flash-frozen in liquid nitrogen and continuous wave EPR spectrum was recorded. Then, the sample was thawed, exposed to UV light (280 nm +/−10 nm, sample positioned side wise and at 40 cm from lamp source, 400 mW received by the sample) during 10 min and flash-frozen again before recording a new EPR spectrum. Controls containing only buffer were also carried out. The illumination system was an Arc Lamp Housing equipped with a xenon-mercury bulb (Newport, USA, model 67005) and connected to an OPS-A150 Arc Lamp power supply (50-500 W) (Newport, USA). The power was set to 350 W. EPR spectra were recorded on a Bruker Elexsys E500 spectrometer operating at X-band at 110 K (BVT 3000 digital temperature controller) with the following acquisition parameters: number of scans, 3; modulation frequency, 100 kHz; modulation amplitude, 5 G; attenuation, 10 dB and microwave power, 20 mW.

### Example 9: Optimized small-scale photobiocatalytic reaction

Optimal illumination conditions are determined by exposing the full reaction mixture to different light intensities (by varying the distance between the light source and the reaction vessel and/or varying the power supply) and different light wavelengths (by using different bandpass filters). Continuous versus intermittent light exposure is also probed, where the duration of intermittent light and “dark” time are varied (from 0 sec to 10 min). An optimal reaction condition is defined as a reaction yielding a stable reaction kinetic and/or a high final product yield (e.g., >70%). The quantity of reaction products is measured off-line by sampling regularly the reaction mixture and stopping the reaction (by either adding EDTA (10 mM final) or acidifying the mixture with HCl (1N final)) prior to quantification. Different CRO/substrate couples are tested, including:
CRO-AlcOx/benzyl alcohol or fatty alcohols (e.g., hexanol)
CRO-GalOx/galactose
CRO-GLOx/glyoxal or methyl glyoxal or glyoxylic acid
CRO-AAO/5-hydroxymethylfurfural (HMF)

In standard reaction conditions, the CRO (10 nM-1 μM) is mixed with the substrate (3-30 mM) in buffered aqueous medium (sodium phosphate (50 mM, pH 8.0) for CRO-AlcOx and CRO-GalOx, sodium 2,2′-dimethylsuccinate (50 mM, pH 6.0) for CRO-GLOx, sodium phosphate (50 mM, pH 7.0) for CRO-AAO). The reaction is carried out at 23° C., under magnetic stirring, in Quartz cuvettes (Hellma, France). The effect of the supply of O₂ is also tested by bubbling through a syringe either air or a mixture of N₂/O₂ (various ratio of O₂ between 0 and 100% v/v), with a gas flow rate of 100 mL/min.

The illumination system is an Arc Lamp Housing equipped with a xenon-mercury bulb (Newport, USA, model 67005) and connected to an OPS-A150 Arc Lamp power supply (50-500 W) (Newport, USA). The power is set to 350 W. A bandpass filter (280±10 nm) with 50 mm optical diameter (Edmund Optics, Lyon) is mounted on the lamp. The light intensity received by the sample is measured outside of the reaction vessel, on the front side facing the light beam, with an optical power meter (Newport, USA).

### Example 10: Photobiocatalytic Reactions Coupled to a Secondary Enzymatic System

Using optimized illumination conditions, we evaluate the effect of secondary enzymatic reactions on the primary photobiocatalytic CRO reaction. A first secondary reaction is the conversion of H₂O₂ produced in situ into H₂O and O₂ by a catalase. Various concentrations of catalase are tested (1 nM-1 μM). Another secondary reaction aims at using H₂O₂ produced in situ as co-substrate of a peroxygenase reaction. In this order, to the photobiocatalytic CRO reaction is added a mixture containing an LPMO (1 μM), cellulose (10 g·L⁻¹Avicel or 0.1% w/v phosphoric acid swollen cellulose) and a reducing agent (100 μM ascorbic acid or cellobiose (3 mM)/cellobiose dehydrogenase (CDH, 0.1-1 μM)). The LPMO is the AA9E from Podospora anserina (PaLPM09E) and the CDH is the CDH from Podospora anserina (PaCDHB), produced and purified as previously described by C. Bennati Granier et al. “Substrate specificity and regioselectivity of fungal AA9 lytic polysaccharide monooxygenases secreted by Podospora anserina,” Biotechnol. Biofuels, vol. 8, no. 1, p. 90, December 2015.

### Example 11: Photobioreactor Upscaling

After establishing the proof-of-concept in 1 mL reactions, the photobiocatalytic reactions is upscaled to 100 mL, using the same illumination system as described above but with a top-wise illumination, with the light source placed at optimal distance from the reaction mixture surface. The reaction is carried out in a 250 mL beaker without spout, under magnetic stirring, at 23° C. The top of the beaker is closed with a home-made Quartz window, equipped with a rubber ring, to allow UV light to reach the solution while minimizing losses by evaporation.

### Example 12: Reaction Mixture Analysis

The CRO-AlcOx activity on benzyl alcohol is determined by quantifying benzaldehyde spectrophotometrically at 254 nm as described above.

The CRO-AlcOx activity on fatty alcohol is determined as follows: 500 μL of the reaction mixture is mixed with 500 μL of cyclohexane/ethyl acetate mixture (1:1), followed by shaking and centrifugation (5 min, 2300 rpm). The organic layer is transferred into a new vial by pipetting and injected in an Optima-σ-3 GC capillary column (30 m×0.25 mm×0.25 μm, Machery-Nagel GmbH&Co KG, Germany) mounted on a gas chromatography (GC)-2014 apparatus (Shimadzu, Japan) equipped with a flame ionization detector (FID). Nitrogen is used as carrier gas, under constant pressure (200 kPa). The inlet and detector temperature are set at 250° C. The temperature gradients of the GC method are described in Table 1. Heptan-1-al (1 mM) is added as internal standard.

The CRO-GalOx activity on galactose is determined in two ways:
For initial qualitative assessment of galactose consumption, we use thin layer chromatography (TLC), where the chromatograms are developed on silica gel plates (Sigma-Aldrich, L'Isle d'Abeau Chesnes, France) with butan-1-ol/acetic acid/H₂O (4:1:1) as the solvent. The plates are dried before immersion in an acidic solution of orcinol (0.1% orcinol (w/v) dissolved in H₂O/ethanol/H₂SO₄ (22:75:3, vol/vol/vol) and visualized by heating at 105° C. for 5 min.
For quantitation, we use high-performance anion exchange chromatography coupled to pulsed amperometric detection (HPAEC-PAD) (ICS-6000 system, ThermoFisher Scientific, Villebon sur Yvette, France). Samples are injected on a CarboPac PA1 (2×50 mm) column operated with 0.1M NaOH (eluent A) at a flow rate of 0.25 ml·min⁻¹ and a column temperature of 30° C. Elution is achieved using a stepwise gradient with increasing amounts of eluent B (0.1 M NaOH, 1 M NaOAc), as follows: 0-10% B over 10 min; 10-30% B over 25 min; 30-100% B over 5 min; 100-0% B over 1 min; and 0% B (reconditioning) for 9 min Chromatograms are recorded using Chromeleon 7.0 software.

The activities of CRO-GLOx on glyoxal/methylglyoxal/glyoxylic acid and CRO-AAO on HMF are analyzed as previously described by M. Daou et al., “Heterologous production and characterization of two glyoxal oxidases from Pycnoporus cinnabarinus,” Appl. Environ. Microbiol., vol. 82, no. 16, pp. 4867-4875, 2016. Briefly, the reaction products are separated on an Aminex HPX-87H column (300×7.8 mm; Bio-Rad) connected to a high pressure liquid chromatography (HPLC) apparatus (Agilent 1260) coupled to a diode-array detector (DAD, monitored wavelengths: 210, 280, 290, 330 and 350 nm). The column temperature is set to 45° C., sulfuric acid (2.5 mM) is used as isocratic eluant with a flow rate of 0.5 ml·min^{-1.} All samples are filtered through 10-kDa-molecular-mass-cutoff Nanosep polyethersulfone membrane columns (Pall Corporation, Saint-Germainen-Laye, France) and 0.45-μm pore-size polyvinylidene difluoride syringe filters (Restek, Lisses, France) before injection in the column.

TABLE 1
GC method

| | Rate | T | Time |
|---|---|---|---|
| Compounds | (° C. · min⁻¹) | (° C.) | (min) |
| Hexanol | — | 40 | 4 |
| | 8 | 100 | 0 |
| | 25 | 220 | 3 |

### Example 13: Light Intensity-Dependent Activity of CgrAlcOx

A solution containing BnOH (3 mM) and CgrAlcOx (10 nM) in aqueous sodium phosphate (50 mM, pH 7.0), at 23° C., was exposed to varying intensities of either UV light (λ=280±10 nm) or UV-Vis broad spectrum light (λ=200-800 nm), under magnetic stirring in air.

Benzyl alcohol was thus oxidized to benzaldehyde. The enzyme activity as a function of the light intensities was measured resulting in the data as shown in FIG. 4, in which the error bars show the standard deviation for 3 independent experiments. 100% intensity (at 280 nm), Iₘₐₓ, corresponds to a light flux (at 4 cm distance) of 1.6 mW·cm⁻², i.e. 36.5 μmol photon·s⁻¹·m⁻².

Thus, 40% intensity, for example, corresponds to a light flux (at 4 cm distance) of 0.64 mW·cm⁻², i.e. 14.6 μmol photon·s⁻¹·m⁻².

It can be seen from FIG. 4 that when using a UV light intensity of 40% (i.e. 14.6 μmol photon·s⁻¹·m⁻²), the photo-activated CgrAlcOx reaches a maximal activity, hence the choice of 40% in subsequent experiments. Furthermore, the use of higher light intensities did not diminish the enzyme rate. In contrast, when using broad UV-Vis light, high light intensities were detrimental for the enzyme rate, with an optimum light intensity of about 10%.

### Example 14: Effect of Duration of Light Exposure on CgrAlcOx Activity

A solution containing benzyl alcohol (3 mM) and CgrAlcOx (10 nM) in aqueous sodium phosphate (50 mM, pH 7.0), at 23° C., was exposed to varying illumination modes under magnetic stirring in air:
continuous UV light (λ=280±10 nm),
discontinuous UV light (λ=280±10 nm), or
discontinuous UV-Vis light broad spectrum (200-800 nm).

The discontinuous mode consisted in on/off illumination cycles of 1/30 min, repeated over 180 min.

A control of CgrAlcOx-catalyzed reaction (in the absence of any activator) gave the residual activity measured in the dark.

Experiments were carried out at 40% or 100% of Iₘₐₓ for UV light and UV-Vis light, respectively. Iₘₐₓ (at 4 cm, λ=280±10 nm)=1.6 mW·cm⁻², i.e. 36.5 μmol photon·s⁻¹·m⁻².

Thus, 40% intensity, for example, corresponds to a light flux (at 4 cm distance) of 0.64 mW·cm⁻², i.e. 14.6 μmol photon·s⁻¹·m⁻².

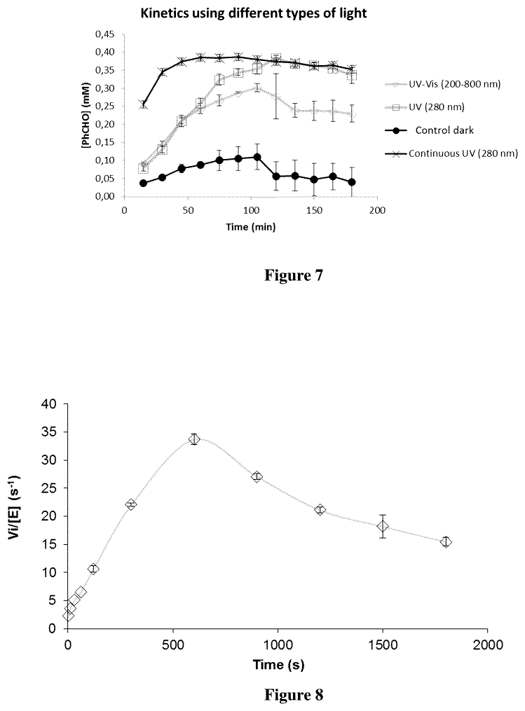

The time-course of benzaldehyde (PhCHO) production was observed as shown in FIG. 7, in which the error bars show the standard deviation for 3 independent experiments.

It can be seen from FIG. 7 that substantially no benzaldehyde was produced in the absence of radiation. When applying radiation, it was observed that higher benzaldehyde yields (>3-fold increase) were obtained. More specifically, the use of discontinuous UV light (280±10 nm) allowed slightly better yields than discontinuous broad UV-Vis light (200-800 nm). Furthermore, in comparison to discontinuous illumination, the use of continuous illumination allowed a much faster initial reaction, reaching a plateau phase similar to the discontinuous UV light experiment. The speed of the reaction can thus be controlled by the illumination mode and the wavelength range.

### Example 15: Effect of Pre-Photoactivation on CgrAlcOx Activity

A solution containing benzyl alcohol (3 mM) and CgrAlcOx (10 nM) in aqueous sodium phosphate (50 mM, pH 7.0), at 23° C. was left in the dark under magnetic stirring in air.

The enzyme had been pre-exposed beforehand to UV light (λ=280±10 nm; 40% of Iₘₐₓ) for varying amounts of time (0 to 1800 s). Iₘₐₓ (at 4 cm, λ=280±10 nm)=1.6 mW·cm⁻², i.e. 36.5 μmol photon·s⁻¹·m⁻².

Thus, 40% intensity corresponds to a light flux (at 4 cm distance) of 0.64 mW·cm⁻², i.e. 14.6 μmol photon·s⁻¹·m⁻².

The results obtained are as shown in FIG. 8, in which the error bars show the standard deviation for 3 independent experiments.

It can be seen from FIG. 8 that the longer the pre-activation time (up to 600 sec), the more active the CgrAlcOx is. Beyond 600 sec of pre-activation, deleterious effects are observed inasmuch as the initial rate of the enzyme decreases.

### Example 16: Effect of Discontinuous Illumination Mode on CgrAlcOx Activity

A solution containing BnOH (3 mM) and CgrAlcOx (10 nM) in aqueous sodium phosphate (50 mM, pH 7.0), at 23° C., was subjected to benzyl alcohol oxidation to benzaldehyde, by exposure, under magnetic stirring, in air, to UV light (λ=280±10 nm; 40% of Iₘₐₓ) with two different discontinuous illumination modes: on/off cycles of either 2/30 min or 10/30 min, repeated over 180 min. Iₘₐₓ (at 4 cm, λ=280±10 nm)=1.6 mW·cm⁻², i.e. 36.5 μmol photon·s⁻¹·m⁻².

Thus, 40% intensity corresponds to a light flux (at 4 cm distance) of 0.64 mW·cm⁻², i.e. 14.6 μmol photon·s⁻¹·m⁻².

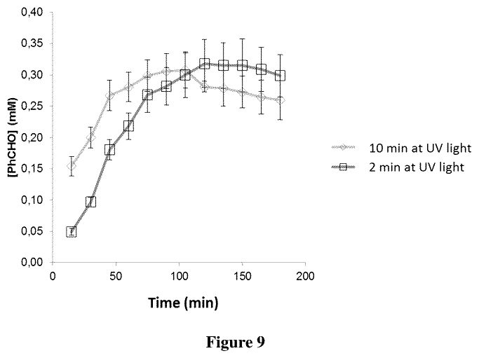

The results obtained are as shown in FIG. 9, in which the error bars show the standard deviation for 3 independent experiments.

It can be seen from FIG. 9 that while the 10/30 min on/off illumination mode leads to faster initial reaction than the 2/30 min illumination mode, in the long term (180 min), the 2/30 min mode is better in terms of product yield. Thus, for this particular application, it appears clear that the optimal illumination mode will depend on the feature sought after, e.g. a fast reaction with a certain final yield, or better yields but with longer reaction times.

### Example 17: Photo-Activation of CgrAAO

CgrAAO-catalyzed oxidation reactions were tested for:
(A) benzyl alcohol (BnOH, 3 mM) into benzaldehyde (PhCHO),
(B) 5-hydroxymethylfurfural (HMF; 3 mM) to 2,5-diformylfuran (DFF), and
(C) 5-hydroxymethyl-2-furan carboxylic acid (HMFCA, 3 mM) to 5-formyl-2-furan carboxylic acid (FFCA).

CgrAAOx (50 nM) was activated by discontinuous UV light (λ=280±10 nm). A control in the dark was also carried out. The discontinuous mode consisted in on/off illumination cycles of 1/30 min, repeated over 180 min.

Experiments were carried out at 40% of Iₘₐₓ. Iₘₐₓ (at 4 cm, λ=280±10 nm)=1.6 mW·cm⁻², i.e. 36.5 μmol photon·s⁻¹·m⁻². Thus, 40% intensity corresponds to a light flux (at 4 cm distance) of 0.64 mW·cm⁻², i.e. 14.6 μmol photon·s⁻¹·m⁻².

All reactions were carried out in an aqueous sodium phosphate solution (50 mM, pH 7.0), at 23° C., under magnetic stirring, in air.

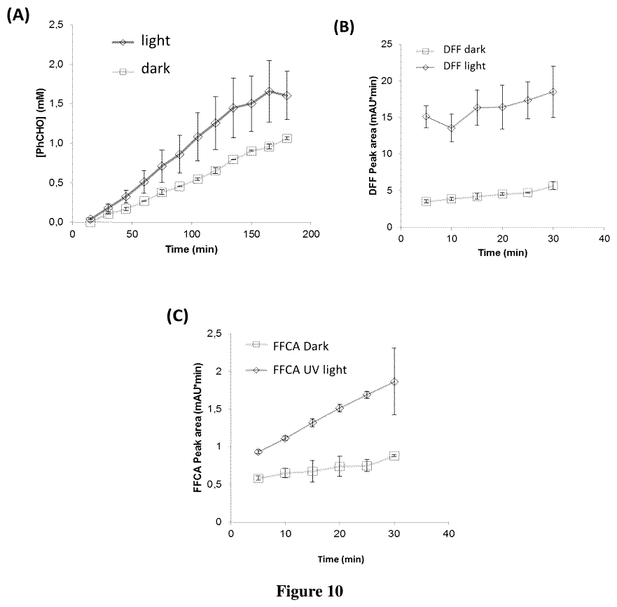

The results obtained for benzyl alcohol oxidation are as shown in FIG. 10(A), in which the error bars show the standard deviation for 2 independent experiments. These results show that CgrAAO can be photo-activated by UV light (280 nm) and yields higher benzaldehyde yields than a non-photo-activated CgrAAO.

The results obtained for 5-hydroxymethylfurfural oxidation are as shown in FIG. 10(B), in which the error bars show the standard deviation for 2 independent experiments. These results confirm that CgrAAO can be photo-activated by UV light (280 nm) and yields higher DFF yields than a non-photo-activated CgrAAO.

The results obtained for 5-hydroxymethyl-2-furan carboxylic acid oxidation are as shown in FIG. 10(C), in which the error bars show the standard deviation for 2 independent experiments. These results confirm that CgrAAO can be photo-activated by UV light (280 nm) and yields higher FFCA yields than a non-photo-activated CgrAAO.

Overall, the results displayed in FIG. 10A-C show that CgrAAO can be photo-activated for different types of reactions, a process that is thus substrate-independent.

### Example 18: Synergy Between CgrAlcOx and Catalase

The time-course of benzaldehyde (PhCHO) production upon BnOH (3 mM) oxidation by CgrAlcOx (10 nM) was determined. Reaction mixture was exposed to discontinuous UV light (λ=280±10 nm), in the absence or presence of catalase (5 nM final). The discontinuous mode consisted in on/off illumination cycles of 2/30 min, repeated over 180 min.

Experiments were carried out at 40% of Iₘₐₓ (λ=280±10 nm). Iₘₐₓ (at 4 cm, λ=280±10 nm)=1.6 mW·cm⁻², i.e. 36.5 μmol photon·s⁻¹·m⁻².

Thus, 40% intensity corresponds to a light flux (at 4 cm distance) of 0.64 mW·cm⁻², i.e. 14.6 μmol photon·s⁻¹·m⁻².

All reactions were carried out in an aqueous sodium phosphate solution (50 mM, pH 7.0), at 23° C., under magnetic stirring, in air.

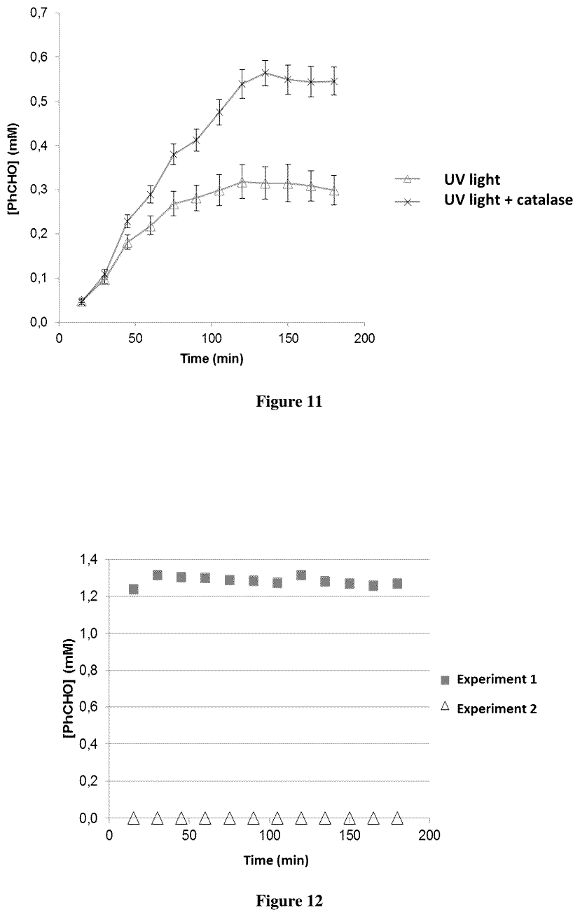

The results obtained are as shown in FIG. 11, in which the error bars show the standard deviation for 3 independent experiments. The results show that there is a clear beneficial effect in terms of benzaldehyde yield upon addition of the catalase to the reaction mixture

### Example 19: Stability of Benzaldehyde and Benzyl Alcohol Under Light Exposure

Benzaldehyde (PhCHO, 1.2 mM) or BnOH (3 mM) were exposed, in the absence of enzyme, to discontinuous UV light (λ=280±10 nm). The discontinuous mode consisted in on/off illumination cycles of 2/30 min, repeated over 180 min.

Experiments were carried out at 40% of Iₘₐₓ (λ=280±10 nm). Iₘₐₓ (at 4 cm, λ=280±10 nm)=1.6 mW·cm⁻², i.e. 36.5 μmol photon·s⁻¹·m⁻².

Thus, 40% intensity, corresponds to a light flux (at 4 cm distance) of 0.64 mW·cm⁻², i.e. 14.6 μmol photon·s⁻¹·m⁻².

All reactions were carried out in an aqueous sodium phosphate solution (50 mM, pH 7.0), at 23° C., under magnetic stirring.

The results obtained are as shown in FIG. 12 and show that benzaldehyde and benzyl alcohol are both stable towards irradiation.

### Example 20: Photoactivation of FgrGalOx

The oxidation of benzyl alcohol (3 mM) to benzaldehyde (PhCHO), and the oxidation of lactose (3 mM) to lactonic acid (LacOx), catalyzed by FgrGalOx (50 nM) were studied.

The reaction mixtures were exposed to discontinuous UV light (λ=280±10 nm), consisting in on/off illumination cycles of 1/30 min.

Experiments were carried out at 40% of Iₘₐₓ (λ=280±10 nm). Iₘₐₓ (at 4 cm, λ=280±10 nm)=1.6 mW·cm⁻², i.e. 36.5 μmol photon·s⁻¹·m⁻².

Thus, 40% intensity, corresponds to a light flux (at 4 cm distance) of 0.64 mW·cm⁻², i.e. 14.6 μmol photon·s⁻¹·m⁻².

All reactions were carried out in an aqueous sodium phosphate solution (50 mM, pH 7.0), at 23° C., under magnetic stirring, in air.

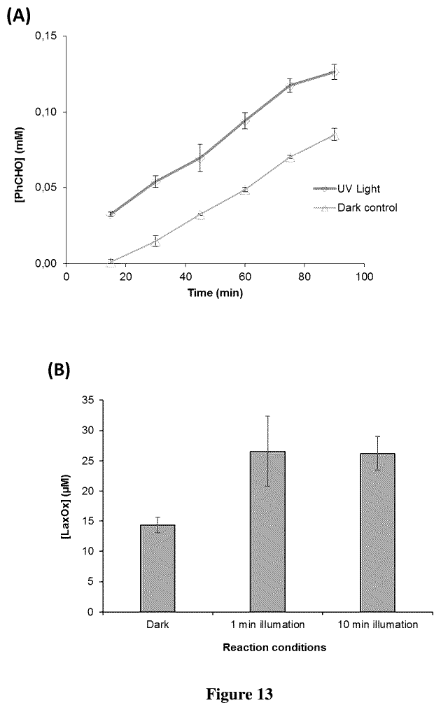

The results obtained for the benzyl alcohol oxidation are as shown in FIG. 13(A), in which the error bars show the standard deviation for 3 independent experiments. The results show a boost in the initial phase of the production of benzaldehyde upon illumination of the FgrGalOx-catalyzed reaction.

The results obtained for the lactose oxidation are as shown in FIG. 13(B), in which the error bars show the standard deviation for 3 independent experiments. The results confirm that FgrGalOx can be photo-activated by UV light as yielding higher LacOx yields than a non-photo-activated FgrGalOx.

1-17.(canceled)

18. A process for the chemical oxidation of an organic compound, said process comprising the step of contacting an organic compound bearing an oxidizable function with at least one Copper-Radical Oxidase (CRO) enzyme in the presence of molecular oxygen,
wherein said at least one CRO enzyme is activated by a step of exposing said at least one CRO enzyme to UV light, to obtain a UV-activated CRO enzyme, whereby the organic compound is oxidized into an oxidized organic product, and whereby hydrogen peroxide is generated.

19. The process for chemical oxidation according to claim 18, wherein the step of exposing said at least one CRO enzyme to UV light is carried out
during the step of contacting,
before the step of contacting, or
both before and during the step of contacting.

20. The process for chemical oxidation according to claim 18, wherein during the contact of said organic compound with said enzyme, the exposure to UV light is continuous or intermittent.

21. The process for chemical oxidation according to claim 18, wherein the UV light has a wavelength comprised from 240 to 320 nm, preferably from 270 to 290 nm, more preferably has a wavelength of about 280 nm.

22. The process for chemical oxidation according to claim 18,
wherein, during the step of exposing said at least one CRO enzyme to UV light, the enzyme is exposed to UV light having a light intensity comprised from 0.01 to 1000 mW/cm², in particular from 1 to 100 mW/cm².
or wherein, during the step of exposing said at least one CRO enzyme to UV light, the enzyme is exposed to of from 1 to 100 μmol photon·s⁻¹·m⁻²; in particular of from 10 to 30 μmol photons⁻¹·m⁻².

23. The process for chemical oxidation according to claim 18, wherein said process comprises between the step exposing said at least one CRO enzyme to UV light before the step of contacting and the step of contacting said UV-activated enzyme with said organic compound,
a step of transfer whereby the organic compound and the UV-activated enzyme are mixed.

24. The process for chemical oxidation according to claim 18, wherein said process is carried out in an aqueous medium, preferably in a buffered aqueous medium, preferably at a temperature comprised between 20 and 50° C. preferably at a temperature of about 23° C.

25. The process for chemical oxidation according to claim 18, wherein the at least one CRO enzyme belongs to the AA5 family, in particular to the AA5_1 or the AA5_2 subfamilies.

26. The process for chemical oxidation according to claim 18,
wherein the at least one CRO enzyme belongs to the AA5_2 subfamily and is an alcohol oxidase (AlcOx), preferably is an alcohol oxidase extracted from Colletotrichum graminicola, in particular having SEQ ID NO: 1, or having at least 60%, in particular at least 70% identity with SEQ ID NO: 1,
or wherein the at least one CRO enzyme belongs to the AA5_2 subfamily and is a galactose oxidase (GalOx) and more preferably is a galactose oxidase extracted from Fusarium graminearum, in particular having SEQ ID NO: 2, or having at least 60%, in particular at least 70% identity with SEQ ID NO: 2,
or wherein the at least one CRO enzyme belongs to the AA5_2 subfamily and is an aryl alcohol oxidase (AAO) and is preferably extracted from Colletotrichum graminicola, in particular having SEQ ID NO: 3, or having at least 60%, in particular at least 70% identity with SEQ ID NO: 3,
or wherein the at least one CRO enzyme belongs to the AA5_1 subfamily and is a glyoxal oxidase (GLOx) and more preferably is a glyoxal oxidase extracted from Pycnoporus cinnabarinus, in particular having SEQ ID NO: 4, or having at least 60%, in particular at least 70% identity with SEQ ID NO: 4,
or wherein the at least one CRO enzyme is a GlxA-type enzyme which is preferably extracted from the bacterium Streptomyces lividans, in particular having SEQ ID NO: 5, or having at least 60%, in particular at least 70% identity with SEQ ID NO: 5.

27. The process for chemical oxidation according to claim 18, wherein said organic compound selected from the group consisting of:
saturated (C₁ to C₂₀) primary alcohols,
unsaturated (C₁ to C₂₀) primary alcohols,
saturated (C₁ to C₂₀) secondary alcohols,
unsaturated (C₁ to C₂₀) secondary alcohols,
(C3 to C₁₀) cyclic alcohols,
aryl alcohols,
heteroaryl alcohols, and
geminal diols,
in particular selected from the group consisting of:
saturated (C₁ to C₂₀) primary alcohols,
allylic alcohols,
aryl alcohols comprising a primary hydroxyl group linked to the aryl group by a Ci alkyl group, and
geminal diols.

28. The process for chemical oxidation according to claim 18, wherein said enzyme is an alcohol oxidase (AlcOx), and wherein said organic compound is:
a primary alcohol and the obtained oxidized organic product is an aldehyde,
an aryl alcohol comprising a primary hydroxyl group linked to the aryl group by a C1 alkyl group, in particular selected from benzyl alcohol, 4-nitrobenzyl alcohol, anisyl alcohol, veratryl alcohol and 4-hydroxybenzyl alcohol, or aryl alcohols comprising an allylic alcohol attached to the aryl group, in particular cinnamyl alcohol,
a saturated, or unsaturated (C1 to C20) primary alcohol, linear or branched, in particular chosen from n-butanol, n-pentanol, n-hexanol or 2,4-hexadiene-1-ol, or
a naturally-occurring polymer comprising long aliphatic chains bearing hydroxyl functions or a sugar, in particular polymers chosen from waxes, cutins and hemicellulose.

29. The process for chemical oxidation according to claim 18, wherein said enzyme is a glyoxal oxidase (GLOx), and wherein said organic compound is:
5-hydroxymethylfurfuryl alcohol, or
lignocellulose derived compounds, in particular glyoxal, methyl glyoxal, glyoxylic acid, formaldehyde or glycerol.

30. The process for chemical oxidation according to claim 18, wherein said enzyme is a galactose oxidase (GalOx), and wherein said organic compound is:
forest and agricultural biomass, in particular fibres, and hemicelluloses, in particular compounds comprising a galactopyranose moiety, more in particular xyloglucan.

31. A method for implementing hydrogen peroxide-driven enzymatic reactions, comprising a step of generating hydrogen peroxide by contacting an organic compound bearing an oxidizable function with at least one Copper-Radical Oxidase (CRO) enzyme in the presence of molecular oxygen,
wherein said at least one CRO enzyme is activated by a step of exposing said at least one CRO enzyme to UV light, to obtain a UV-activated CRO enzyme,
whereby the organic compound is oxidized into an oxidized organic product,
and whereby hydrogen peroxide is generated.

32. The method according to claim 31, wherein the hydrogen peroxide-driven enzymatic reaction is selected from the group
consisting of decarboxylations, hydroxylations, halogenations, epoxidations, sulfoxidations and Baeyer-Villiger oxidations,
or wherein the hydrogen peroxide-driven enzymatic reaction consists in the enzymatic conversion of said hydrogen peroxide into oxygen and water, in particular using a catalase enzyme,
or wherein the hydrogen peroxide-driven enzymatic reactions consists in the degradation of a polysaccharide, said reaction comprising contacting said polysaccharide with one or more lytic polysaccharide monooxygenase (LPMO), in the presence of an external source of electrons, said source of electrons being in particular a reducing agent.


SEQUENCE LISTING (5 sequences)

SEQ ID NO: 1 - protein, 506 aa, Colletotrichum graminicola
  misc_feature: Alcohol oxidase (AlcOx)
MPTLRSALRNLPAALLALAAACEAQNVGKWGPMVKFPVVPVAVALVPETGNLLVWSSGWPNRWTTAGNGKTYTSLYNVNTGNISDAIVQNTQHDMFCPGTSLDADGRIIVTGGSSAAKTSVLDFKKGESSPWTPLSNMQISRGYQSSCTTSEGKIFVIGGSFSGAGTRNGEVYDPKANTWTKLAGCPVKPLVMQRGMFPDSHAWLWSWKNGSVLQAGPSKKMNWYDTKGTGSNTPAGLRGTDEDSMCGVSVMYDAVAGKIFTYGGGKGYTGYDSTSNAHILTLGEPGQAVQVQKLANGKYNRGFANAVVMPDGKIWVVGGMQKMWLFSDTTPQLTPELFDPATGSFTPTTPHTVPRNYHSTALLMADATIWSGGGGLCGANCKENHFDGQFWSPPYLFEADGVTPAKRPVIQSLSDTAVRAGAPITITMQDAGAYTFSMIRVSATTHTVNTDQRRIPLDGQDGGDGKSFTVNVPNDYGVAIPGYYMLFAMNEAGVPCVAQFFKVTL

SEQ ID NO: 2 - protein, 680 aa, Fusarium graminearum
  misc_feature: Galactose oxidase (GalOx)
MKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 3 - protein, 711 aa, Colletotrichum graminicola
  misc_feature: Aryl alcohol oxidase (AAO)
MVRSCAYKAIAAASLLSQLASAAITSCPNNETVWETPIGVKYTLCPGSDYQNGGASLQTVRDIQSSLECAKICDSDARCNRAVYDNVNKACDVKNSTNPMQWAADDRFETIRLTNDLPEGAFISTCSFNETSYRVPETNAEYRICPDTDYTGVNAKVVEGVTTIQACAELCSNTQDCRKSVFDHINNACAIKAAEPATSIFWVQDKQFSTIRLPENIDPAVKGKWGDLIRLPVIPVAAYIVPSYPEPSRLLFFSSWSNDAFSGASGMTQFGDYDFATGAISQRTVTNTHHDMFCPGISQLEDGRILIQGGSDADTVSIYDPATNEFTRGPNMTLARGYQTSCTLSNGKVFTIGGAYSGERVGKNGEVYDPVANAWTYLPGADFRPMLTNDHEGIWREDNHAWLFGWKNGSIFQAGPSKDQHWYGIQGNGTVAKAATRDDDDAMCGVWVMYDAVAGKIFSAGGSPDYTDSPATQRAHITTIGEPNTPAEVERVADMGFPRGFANAVVLPDGQVLVTGGQRMSLVFTNTDGILVAELFNPETREWKQMAPMAVPRNYHSVSILLPDATVFSGGGGMCWVQNVGDSTAGCDKTVDHSDGEIFEPPYLFNEDGSRAARPVISAISADPIKAGATLTFTVEGVEGQGTAALIRLGSVTHSVNSDQRRVPLNVTVSGNEYSATLPDDYGILLPGYYYLFVSTPQGTPSIAKTVHVIL

SEQ ID NO: 4 - protein, 559 aa, Pycnoporus cinnabarinus
  misc_feature: Glyoxal oxidase (GLOx)
MFQTTLHLLFVLVVTGRGLAAPSTPTGWQFNLKAERSGIVALESIVVSPTLVVFFDRATNDPLQINNHSAWGALWNLETSTVRALDVLTNSFCASGALLSNGTMASIGGDPNGFPGNPAIHPGTQAIRLFEPCDSPTGEGCTLFEDPVTLHLLEKRWYPSSVRIFDGSLLIVGGMHEETPFYNTDPALSFEFFPPKESTPRPSEFLNRSLPANLFPRVFALPDGKVFMVANNQSIIYDIEANTERILPDIPNNVRVTNPIDGSAILLPLSPPDFVPEVLVCGGTQTDTIDPSLLTSQTPASSQCSRIRLDEEGIARGWEVEHMLEGRMMPELVHLPNGQVLIANGARTGFAAIASVSDPVGGSNADHAVLVPSLYTPDAPLGTRISNVGLPSSGIARVYHSSITLTPQGNFLIAGSNPNNNSSVTAGVKFPSEFRVQTLDPPFMFVERPKILSMPKKLAFGKSFTVPIAVPSTLAHPGAKVQVSLMDLGFSSHAFHSSARLVFMNAKISQDGKSLTFTTPPNGRVYPPGPATIFLTIDDVTSEGAWVMMGSGNPPPTLE

SEQ ID NO: 5 - protein, 644 aa, Streptomyces lividans
  misc_feature: GlxA-type enzyme
MKDRAGRRRARRFAIGTAVVVALAGMNGPWLYRFSTEKYHQYKINQPEYKAANGKWEIIEFPEKYRQNTIHAALLRTGKVLMVAGSGNNQDNSDDKQYDTRIWDPVKGTIKKVPTPSDLFCTGHTQLANGNLLIAGGTKRYEKLKGDVTKAGGLMVVHNENPDKPITLPAGTKFTGKENGKTFVSKDPVLVPRAEKVFDPATGAFVRNDPGLGRIYVEAQKSGSAYETGTEDNYRVQGLSGADARNTYGIAQKLALDKKDFQGIRDAFEFDPVAEKYIKVDPMHEARWYPTLTTLGDGKLSVSGLDDIGQLVPGKNEVYDPKTKAWTYTDKVRQFPTYPALFLMQNGKIFYSGANAGYGPDDVGRTPGVWDVETNKFTKVPGMSDANMLETANTVLLPPAQDEKYMVIGGGGVGESKLSSEKTRIADLKADDPKFVDGPSLEKGTRYPQASILPDDSVLVSGGSQDYRGRGDSNILQARLYHPDTNEFERVADPLVGRNYHSGSILLPDGRLMFFGSDSLYADKANTKPGKFEQRIEIYTPPYLYRDSRPDLSGGPQTIARGGSGTFTSRAASTVKKVRLIRPSASTHVTDVDQRSIALDFKADGDKLTVTVPSGKNLVQSGWYMMFVTDGEGTPSKAEWVRVP